(19) United States
(12) Patent Application Publication
(10) Pub. No.: US 2026/0325694 A1
(43) Pub. Date: Oct. 22, 2026

(54) Chitosan-copper hydrogel, methods of production thereof, compositions comprising it, methods using it, a surface of an object, fabric, non-woven fabric covered with it and uses of the chitosan-copper hydrogel

(71) Applicant: Instytut Biochemii i Biofizyki PAN, Warszawa (PL)

(72) Inventors: Krzysztof DRABIKOWSKI, Warszawa (PL); Wojciech BAL, Wilcza Gora (PL)

(21) Appl. No.: 19/482,118

(22) PCT Filed: May 6, 2024
(86) PCT No.: PCT/PL2024/050030

§ 371 Date: Nov. 6, 2025

(30) Foreign Application Priority Data
May 7, 2023 (PL) P.444765

(51) Int. Cl. A61L 2/18; A01N 25/04; A01N 63/14; A61L 101/42; A61L 101/50; A61L 103/00

(52) U.S. Cl. CPC A61L 2/18; A01N 25/04; A01N 63/14; A61L 2101/42; A61L 2101/50; A61L 2103/00

(57) ABSTRACT

The invention relates to a chitosan-copper hydrogel containing from 0.0008 mM to 300 mM of chitosan with a degree of chitosan deacetylation of at least 20%, saturation with copper(II) ions in an amount corresponding to from 5 to 100% of the total content of the reactive amino groups of chitosan and pH 7±1, which has a liquid form at the temperature above 80° C.

The invention also relates to a method of production of chitosan-copper hydrogel, a method for covering a surface with chitosan-copper hydrogel, a surface, a fabric, a non-woven fabric covered with it, a pharmaceutical, veterinary, cosmetic or care composition comprising it, a composition with chitosan-copper hydrogel for covering surfaces in order to prevent the development, to inhibit the growth and kill bacteria, fungi, viruses, protozoa, various uses of chitosan-copper hydrogel including as a biocidal agent, antiseptic agent, antimicrobial agent, bacteriostatic agent, antiviral agent, fungicidal agent, an agent for covering, soaking and disinfection of surfaces, as well as a method for purifying aqueous solutions from microbiological contaminants using chitosan-copper hydrogel.

### TECHNICAL FIELD

An object of the invention is a chitosan-copper hydrogel, which is a complex of chitosan with copper(II) ions. The invention also relates to methods of production of chitosan-copper hydrogel, a method for covering a surface with chitosan-copper hydrogel, a surface, a fabric, a non-woven fabric covered with it, a pharmaceutical, veterinary, cosmetic or care composition comprising it, a composition with chitosan-copper hydrogel for covering surfaces in order to prevent the development, inhibit the growth and kill bacteria, fungi, viruses, protozoa (protista), the invention also relates various uses of chitosan-copper hydrogel including as a biocidal agent, antiseptic agent, antimicrobial agent, bacteriostatic agent, antiviral agent, fungicidal agent, an agent for covering, soaking and disinfection of surfaces, as well as the invention also relates a method for purifying aqueous solutions from microbiological contaminants using chitosan-copper hydrogel.

The hydrogel formulation of chitosan with copper(II) ions is therefore intended for the production of agents that protect against infectious agents, especially bacteria, viruses, fungi and protozoa.

### STATE OF ART

Metallic copper, as well as free and complexed copper ions, have established biocidal activity, including antibacterial, antiviral, antifungal and antiparasitic, as discussed, i.a., in G. Borkow, J. Gabbay, Curr Med Chem 12, 2163-2175, 2005. As described in M. G. Schmidt et al. Appl Environ Microbiol 86, e01886-19, 2020, replacing steel surfaces in healthcare facilities by copper, or containing copper, preferably in the form of oxides, significantly reduces the level of drug-resistant bacteria in the healthcare facility environment. As shown in N. van Doremalen et al. N Engl J Med 382, 1564-1567, 2020, the surface of metallic copper inactivates SARS-Cov-2 viruses. Nevertheless, the widespread use of surfaces and chemical compounds releasing copper, such as metallic copper as a subject to a natural erosion process, leads to the development of microorganisms resistant to the biocidal effect of copper, see C. Espirito Santo et al., Appl Environ Microbiol 76, 1341-1348, 2010. Moreover, obtaining materials, e.g., fibers, containing biocidal active copper may be a complicated, multi-stage process, requiring the use of dangerous reagents and expensive, see G. Borkow, J. Gabbay, FASEB J 18, 1728-1730, 2004.

In the state of the art, there are not known permanent methods of applying copper salts to surfaces, especially on fabrics and non-woven fabrics, because the obtained surface is mechanically unstable and non-uniform. Zhang et al. described a method of applying a copper hydroxide-cellulose adduct to a chemically activated polyester material and using such a material for phase separation in water-oil emulsions. No biological properties of such an adduct have been disclosed (Z. Zhang et al. Chemosphere 302, 134840, 2022).

Chitosan is a linear polysaccharide composed of randomly distributed D-glucosamine and N-acetyl-D-glucosamine units linked in the β-(1-4) position, obtained from animal or fungal chitin. Methods for producing chitosan by processing chitin are known in the art. The purity of chitosan is described by the degree of deacetylation, i.e. the content of D-glucosamine units.

Chitosan has many applications in the cosmetics industry and medicine. Due to the not very strong biocidal effect of chitosan, weakened in a neutral environment, a number of modifications by small molecules forming covalent bonds with its oxygen or nitrogen atoms have been described to increase its solubility and biocidal properties. Hydrogels made of chitosan using additional cross-linking substances, other polymers, as well as calcium ions are known in the art and serve to deliver bioactive substances, e.g. antibiotics, see C.-L. Ke, et al. Polymers 13, 904, 2021.

Chitosan is a completely non-toxic compound, so its use, even on an industrial scale, does not cause environmental pollution. An important feature of chitosan is its full biocompatibility. Further advantages include high sorption capacity, high chemical reactivity and the ability to chelate metals due to the presence of numerous anchoring groups on the surface. In an aqueous environment, chitosan can interact with metal ions to form coordination bonds and hydrogen bonds. However, it is believed that chemical modification of chitosan is necessary to obtain effective binding of metal ions, e.g. for applications in environmental treatment, see C. Verma, M. A. Quraishi, Curr Res Green Sust Chem 4, 100184, 2021.

Chitosan is a polymer of glucosamine. The ability of glucosamine to bind copper(II) ions is known in the art. The article by Micera et al. Inorg. Chim. Acta 107, 45-48, 2005 presents the structure of complexes formed by glucosamine with copper(II) ions, forming a coordination bond between the Cu²⁺ ion and the nitrogen atoms of one (in a weakly acidic environment) or two glucosamine molecules (in a neutral and alkaline environment) and with an adjacent oxygen atoms of these molecules (this structure is shown in FIG. 1 A1). Moreover, the described complexes have low thermodynamic stability. The formation of a complex of glucosamine with the copper(II) ion in a neutral environment is characterized by an absorption band with a maximum at 640 nm. However, the hydroxyl group in position 1 of glucosamine, which is involved in the binding of the copper(II) ion, cannot serve this function in chitosan because it is blocked by the formation of a glycosidic bond, which is responsible for the formation of the chitosan polymer. Thus, the state of the art did not indicate that chitosan had the ability to effectively bind copper(II) ions.

The bactericidal activity of the chitosan complex with copper(II) ions has been described (Mekahlia and Bouzid, Physics Procedia (2009) 2, 1045-1053). However, the described process takes place in the environment of hydrochloric acid, and hydrochloric acid itself has biocidal properties. The absence of a complex of chitosan with copper(II) ions in the hydrochloric acid environment is evidenced by the absorption spectrum of the complex in the visible range given in the publication, which corresponds to the spectrum of copper(II) aqua-ion, which is not chemically bonded to chitosan.

The synthesis and fungicidal activity of nanoparticles composed of chitosan and copper(II) ions have been described (Saharan et al. Int J Biol Macromol 75, 346-353, 2015). These nanoparticles contain large amounts of anionic polyphosphates that cross-link chitosan by interacting with positively charged amino groups, which excludes the formation of stable copper(II) complexes. Non-specific occlusion of copper(II) ions was proposed in the hypothetical structure of nanoparticles suggested by the authors. These nanoparticles contain one copper(II) ion per approximately 20 chitosan monomers.

From the description PL233402 B1 a composition containing chitosan, sulfate ions and phosphate ions is known, intended for the removal of Cu²⁺ ions from water by precipitation.

From the description EP1512773 a coating of metal medical devices with chitosan is known, in order to make them biocompatible and to prevent irritation of the body by metal ions coming from these devices.

From the description CN106146913A formation a chitosan hydrogel with an initial pH 6.2 with the addition of metal ions only at particularly described molar ratios is known. In particular, the chitosan-copper hydrogel according to this description is obtained for the proportion: 4 parts of chitosan and 10 parts of copper(II) nitrate, and therefore with an excess of copper over chitosan (over 100%). It is obvious to a person skilled in the art that the pH of the final product obtained according to CN106146913A is lower than the initial pH 6.2 given therein, because the addition of Cu(II) salt lowers the pH because by binding to the amino groups of chitosan the Cu(II) ions displace hydrogen ions from them. It follows from the description CN106146913A that the final pH value of such a hydrogel is significantly below pH 6. Moreover, Cu(II) saturation above 100% does not cause the breakdown of the hydrogel from CN106146913A, which distinguishes it from the chitosan-copper hydrogel according to the present invention. The description CN106146913A does not disclose any biocidal properties of the substance described therein.

A hydrogel containing chitosan and Cu(II) ions has been described, with bactericidal properties against the bacteria Pseudomonas aeruginosa, which is produced with the obligatory use of ammonia (Li et al. Int J Biol Macromol 133, 67-75, 2019). According to the description, exposing the mixture of chitosan and copper(II) nitrate to ammonia for 12 hours is necessary to obtain the hydrogel. The publication indicates that the reaction is carried out for 12 hours to produce a chitosan-copper-ammonia hydrogel, i.e. CTS-Cu²⁺/NH₃, where CTS stands for chitosan (page 68, pt 2.2) with the structure shown in drawing 1B, according to which each Cu(II) ion is bound to two nitrogen atoms (herein described as FIG. 1 A2), which is different from the hydrogel described in the present invention. Therefore, the hydrogel prepared according to Li et al. (2019) is a chitosan-copper-ammonia hydrogel, not a chitosan-copper hydrogel, because ammonia remains in the hydrogel structure and forms bonds with cupric ions. However, the biocidal effect of the hydrogel according to Li et al. against P. aeruginosa is based on the release of free cupric ions from the chitosan-copper-ammonia hydrogel, which distinguishes the mechanism of action from the mechanism described in the present invention of the chitosan-copper hydrogel. Such release is enabled only by the binding of the Cu(II) ion within the hydrogel to ammonia. From the description CN114805859A, a chitosan hydrogel with the addition of metal ions, including copper, is known, produced by heating a solution containing chitosan and a metal salt to a temperature above 80° C. and below 100° C., in particular 85° C. The substance according to this description has hydrogel properties only at high temperatures in the stated range between 80° C. and 100° C. Lowering the temperature below 80° C., including to the room temperature, reversibly transforms the described substance into liquid state, which distinguishes it from the chitosan-copper hydrogel according to the present invention, which is in a liquid form at the temperature above 80° C. Therefore, the structure of the chitosan hydrogel with the addition of metal ions known from the description CN114805859A is different than the structure of the chitosan-copper hydrogel according to the invention. The description CN114805859A does not disclose any biocidal properties of the substance described there.

There are no known hydrogels in the state of the art containing chemically or physically unmodified chitosan and copper(II) ions for covering surfaces colonized by bacteria, fungi, viruses or protozoa, or for use as additives to an environment containing bacteria, fungi, viruses or protozoa in order to neutralize them. Moreover, all the described materials containing biocidal copper(II) ions act by releasing these ions into the environment containing the above pathogens, which may contribute to the increase and spread of resistance of these pathogens to the biocidal effect of copper.

### Essence of the Invention

The aim of the invention is therefore to provide an improved, environmentally neutral and biocompatible, and therefore substantially non-toxic, biocidal agent capable of immobilizing, preventing, inhibiting the growth and killing of bacteria, fungi, viruses or protozoa, both in solution and on surfaces coated with it, particularly as an biocidal agent for use in medicine and veterinary medicine and, at the same time, easy to produce. The aim is to provide a biocide with a broad spectrum of activity and properties enabling its various uses, especially for covering and saturating surfaces with it, especially those that require microbiological cleanliness, so as to prevent the development or colonization by bacteria, fungi, viruses or protozoa.

This aim was achieved by creating a hydrogel composed of chitosan chains and copper(II) ions with high adhesion to the substrate, capable of essentially permanently bonding to the substrate coated with it, with biocidal properties, at the same time exhibiting a broad and universal spectrum of activity against various microorganisms, including bacteria, viruses and fungi and protozoa. The aim was achieved by providing practical medical and non-medical applications of chitosan-copper hydrogel and by providing optimized, simple and adapted to various surfaces methods of its production and covering such surfaces.

The invention relates to a chitosan-copper hydrogel that contains from 0.0008 mM to 300 mM of chitosan with a degree of chitosan deacetylation of at least 20%, the degree of deacetylation quantitatively corresponds to the content of the reactive amino groups of chitosan; wherein the reactive amino groups of chitosan are saturated with copper(II) ions in an amount corresponding to from 5 to 100% of the total content of reactive amino groups of chitosan; the chitosan-copper hydrogel has pH 7±1, preferably it has pH 7, and the chitosan-copper hydrogel has a liquid form at the temperature above 80° C.

A preferred chitosan-copper hydrogel is produced by basic subunits I, basic subunits II, basic subunits III, wherein the basic subunits I are non-deacetylated glucosamine subunits of chitosan of formula (I)

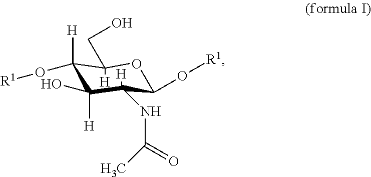

wherein the basic subunits II are deacetylated chitosan glucosamine subunits with reactive chitosan amino groups unsaturated with copper(II) ions of the formula (II)

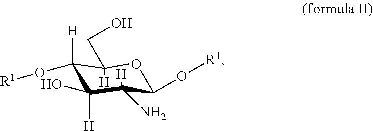

wherein the basic subunits III are deacetylated chitosan glucosamine subunits with reactive chitosan amino groups saturated with copper(II) ions of the formula (III)

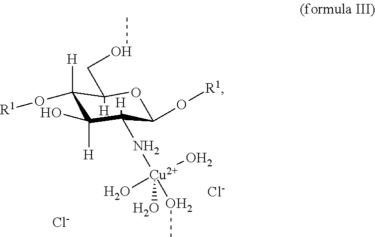

wherein the basic subunits I, II, III are linked together in any order, and wherein symbol R¹ in the formula I, II, III stands for a chain composed of these basic subunits I, II, III.

In the chitosan-copper hydrogel, the basic subunits I, II, III are linked to each other in any order, in quantities described by the hydrogel composition parameters: degree of deacetylation (II+III)/(I+II+III)×100% and degree of saturation with cupric ions (III)/(II+III)×100%, and the symbol R1 stands for chains composed of these subunits. The chain length depends on the length of the chitosan chains.

The same structure described by the basic subunits I, II, III linked to each other in any order characterizes the chitosan-copper hydrogel produced by the methods for producing the chitosan-copper hydrogel according to the invention presented below.

The chitosan-copper hydrogel preferably contains from 0.6 mM to 180 mM of chitosan.

In a preferred chitosan-copper hydrogel, the degree of chitosan deacetylation is at least 30%, 40%, 50%, 60%, 70%, 80%, 90%, 95%, 98%, 99%, most preferably 100%.

In a preferred chitosan-copper hydrogel, the amino groups of chitosan are saturated with copper(II) ions in an amount corresponding to from 10 to 100%, preferably from 20% to 95%, preferably from 30 to 90%, preferably from 40 to 80%, preferably from 50 to 100%, most preferably from 50 to 70%, saturation of the reactive amino groups of chitosan.

The chitosan-copper hydrogel is preferably in dried form, preferably in freeze-dried form, preferably in the form of powder, granulate, preferably in dried form ready to be reconstituted by adding water.

The invention also relates to the first method of preparing a chitosan-copper hydrogel, including the steps in which a) chitosan with a degree of deacetylation of at least 20%, the degree of deacetylation corresponds quantitatively to the content of reactive amino groups of chitosan, dissolved in inorganic and/or organic acid soluble in water at a concentration of 0.1% to 10% weight-volume (w/v), preferably 1% w/v of the acid to obtain a chitosan solution with a concentration of 0.0008 mM to 300 mM; preferably, dissolution of chitosan is carried out at 10-80° C., preferably at 20-30° C.; b) the solution of dissolved chitosan from step a) is separated from the acid and adjusted to pH 7±1 by dialysis in water, preferably distilled water, preferably at the temperature of 20-30° C.; c) an aqueous solution of inorganic or organic copper(II) salt is added to the solution from step b) while stirring vigorously, and the copper(II) salt solution is added in an amount corresponding to a molar ratio in terms of copper(II) ions corresponding to from 5 to 100% saturation of the reactive amino groups of chitosan, preferably at a temperature of 20-30° C., wherein the produced chitosan-copper hydrogel has pH 7±1, preferably has pH 7, and wherein the chitosan-copper hydrogel has a liquid form at the temperature above 80° C. In the preferred first method of preparing a chitosan-copper hydrogel in step a) from 0.6 mM to 180 mM of chitosan is used.

In the preferred first method of preparing the chitosan-copper hydrogel in the step a) chitosan with a degree of deacetylation of at least 30%, 40%, 50%, 60%, 70%, 80%, 90%, 95%, 98%, 99%, most preferably 100% is used.

In the preferred first method of preparing the chitosan-copper hydrogel in the step d) an aqueous solution of an inorganic or organic copper(II) salt is added in an amount corresponding to a molar ratio in terms of copper(II) ions, corresponding to from 10 to 100%, preferably from 20% to 95%, preferably from 30 to 90%, preferably from 40 to 80%, preferably from 50 to 100%, most preferably from 50 to 70% saturation of the reactive amino groups of chitosan.

In the preferred first method of preparing the chitosan-copper hydrogel in step a) the acid is an inorganic acid selected from hydrochloric acid, sulfuric acid, nitric acid, perchloric acid and/or an organic acid selected from carboxylic acid, hydroxycarboxylic acid, sulfonic acid, acetic acid, trifluoroacetic acid, glycolic acid, lactic acid, mandelic acid, formic acid, salicylic acid, or a mixture thereof, more preferably the acid is acetic acid.

In the preferred first method of preparing the chitosan-copper hydrogel in the step c) the aqueous solution of the inorganic or organic copper(II) salt is an aqueous solution of copper(II) chloride, sulfate, nitrate, perchlorate, acetate, trifluoroacetate, lactate, glycolate, formate, salicylate or mandelate.

The invention also relates to the second method for producing a chitosan-copper hydrogel, including steps in which a) chitosan with a degree of chitosan deacetylation of at least 20%, wherein the degree of deacetylation corresponds quantitatively to the content of the reactive amino groups of chitosan, dissolved in a water-soluble inorganic and/or organic acid at a concentration of 0.1% to 10%, w/v, preferably 1% w/v of acid, to obtain a chitosan solution of 0.05 mM to 300 mM; wherein, preferably, from 0.6 mM to 180 mM of chitosan is used; wherein, preferably, chitosan with a degree of deacetylation of at least 30%, 40%, 50%, 60%, 70%, 80%, 90%, 95%, 98%, 99%, most preferably 100% is used; wherein, preferably, the acid is an inorganic acid selected from hydrochloric acid, sulfuric acid, nitric acid, perchloric acid and/or an organic acid selected from carboxylic acid, hydroxycarboxylic acid, sulfonic acid, acetic acid, trifluoroacetic acid, glycolic acid, lactic acid, mandelic acid, formic acid, salicylic acid, or a mixture thereof, more preferably the acid is acetic acid, wherein the dissolution of chitosan is preferably carried out at the temperature of 10-80° C., preferably at the temperature of 20-30° C.;
b) an aqueous solution of an inorganic or organic copper(II) salt is added to the solution from the step a) while stirring, and the copper(II) salt solution is added in an amount corresponding to from 5 to 100% of saturation of the reactive amino groups of chitosan, preferably at the temperature of 20-30° C.; more preferably, an aqueous solution of an inorganic or organic copper(II) salt is added in an amount corresponding to from 10 to 100%, preferably from 20% to 95%, preferably from 30 to 90%, preferably from 40 to 80%, preferably from 50 to 100%, most preferably from 50 to 70% of saturation of the reactive amino groups of chitosan; more preferably, the aqueous solution of an inorganic or organic copper(II) salt is an aqueous solution of copper(II) chloride, sulfate, nitrate, perchlorate, acetate, trifluoroacetate, lactate, glycolate, formate, salicylate, or mandelate;
c) the mixture obtained in the step b) is contacted with a solution of a base or a buffer not containing agents chelating cupric ions, preferably the base is added to the solution to obtain pH 7±1, more preferably pH 7, preferably the base is selected from NaOH, KOH, preferably at the temperature of 20-30° C., wherein the produced chitosan-copper hydrogel has pH 7±1, preferably has pH 7, and wherein the chitosan-copper hydrogel has a liquid form at the temperature above 80° C.

In the preferred second method of producing the hydrogel, the chitosan-copper hydrogel is formed in situ on the surface to be covered with the chitosan-copper hydrogel, wherein between the steps b) and c) the mixture prepared in the step b) is contacted with the surface on which the hydrogel is to be produced, preferably the surface is selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface is hydrophilic and/or negatively charged, and the contacting leads to by applying, spreading, spraying and/or immersing the surface in the mixture prepared in the step b).

The invention also relates to the third method for producing chitosan-copper hydrogel, including steps in which a) chitosan with a degree of chitosan deacetylation of at least 20%, wherein the degree of deacetylation corresponding quantitatively to the content of the reactive amino groups of chitosan; is dissolved in a water-soluble inorganic and/or organic acid at a concentration of 0.1% to 10% w/v, to obtain a solution of 0.0008 mM to 300 mM; wherein from 0.6 mM to 180 mM of chitosan is preferably used; wherein chitosan with a degree of deacetylation of at least 30%, 40%, 50%, 60%, 70%, 80%, 90%, 95%, 98%, 99%, most preferably 100% is used; wherein, preferably, the acid is an inorganic acid selected from hydrochloric acid, sulfuric acid, nitric acid, perchloric acid and/or an organic acid selected from carboxylic acid, hydroxycarboxylic acid, sulfonic acid, acetic acid, trifluoroacetic acid, glycolic acid, lactic acid, mandelic acid, formic acid, salicylic acid, or a mixture thereof, more preferably it is acetic acid; wherein the dissolution of chitosan is preferably carried out at the temperature of 10-80° C., preferably at the temperature of 20-30° C.;
b) to the solution obtained in a) a solution of base or buffer not containing agents chelating cupric ions is added to obtain pH 7±1, preferably the base is selected from NaOH, KOH, preferably at the temperature of 20-30° C.;
c) the mixture obtained in step b) is contacted with, preferably added to, an aqueous solution of an inorganic or organic copper(II) salt while stirring, and the copper(II) salt solution is added in an amount corresponding to from 5 to 100% of saturation of the reactive amine groups of chitosan; preferably at the temperature of 20-30° C.; more preferably, an aqueous solution of an inorganic or organic copper(II) salt is added in an amount corresponding to from 10 to 100%, preferably from 20% to 95%, preferably from 30 to 90%, preferably from 40 to 80%, preferably from 50 to 100%, most preferably from 50 to 70% of saturation of the reactive amino groups of chitosan; more preferably, the aqueous solution of an inorganic or organic copper(II) salt is an aqueous solution of copper(II) chloride, sulfate, nitrate, perchlorate, acetate, trifluoroacetate, lactate, glycolate, formate, salicylate, or mandelate wherein the produced chitosan-copper hydrogel has pH 7±1, preferably has pH 7, and wherein the chitosan-copper hydrogel has a liquid form at the temperature above 80° C.

In the preferred third method of producing the hydrogel, the chitosan-copper hydrogel is produced in situ on the surface to be covered with the chitosan-copper hydrogel, and between the steps b) and c) the mixture produced in the step b) is contacted with the surface on which the hydrogel is to be produced, wherein the surface is preferably selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface is hydrophilic and/or negatively charged in nature, and contacting is carried out by applying, spreading, spraying and/or immersing the surface in the mixture prepared in the step b). The invention also relates to a method of covering a surface with a chitosan-copper hydrogel of the invention and/or produced by any indicated method for producing the chitosan-copper hydrogel of the invention, wherein the surface being covered is contacted with the chitosan-copper hydrogel, wherein the contacting is carried out by overlaying, applying, spraying the chitosan-copper hydrogel onto the surface and/or immersing the surface in the chitosan-copper hydrogel, and then drying the surface covered with the chitosan-copper hydrogel; wherein the surface is preferably selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface has a hydrophilic character.

In a preferred method, a surface is covered with hydrogel, the chitosan-copper hydrogel is heated to the temperature of 40-100° C., preferably 50-80° C., preferably 50° C. before contacting the surface to be covered.

The invention also relates to a surface of an object covered with a chitosan-copper hydrogel of the invention and/or produced by any indicated method for producing a chitosan-copper hydrogel of the invention, and/or covered according to a surface covering method with a chitosan-copper hydrogel of the invention, the surface of the object is preferably selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface has a hydrophilic and/or negatively charged character.

The invention also relates to a fabric or non-woven fabric which is covered with a chitosan-copper hydrogel of the invention and/or produced by any indicated method for producing a chitosan-copper hydrogel of the invention, and/or covered according to a surface covering method with a chitosan-copper hydrogel of the invention, wherein the fabric or non-woven fabric is made of cotton, linen, wool, silk, polyester, polylactide, aromatic-aliphatic polyesters, polystyrene or a mixture thereof.

The invention also relates to compositions for covering surfaces to prevent the development, inhibit the growth and kill bacteria, fungi, viruses, protozoa which contains a chitosan-copper hydrogel of the invention and/or prepared by any indicated method for producing a chitosan-copper hydrogel of the invention, wherein the surface is preferably selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface has a hydrophilic character.

The invention also relates to a pharmaceutical composition which contains a chitosan-copper hydrogel of the invention and/or prepared by any method for producing a chitosan-copper hydrogel of the invention, the composition further comprises at least one pharmaceutically acceptable carrier.

The pharmaceutical composition is preferably in the form of a liquid, emulsion, gel, spray, foam, nasal spray, mouthwash, hand wash gel, wet wipe or dressing, preferably the dressing is in the form of a patch with hydrogel, gauze with the composition applied, hydrocolloid dressing, hydrofibrous dressing, bandage with the composition applied.

The invention also relates to a pharmaceutical composition comprising a chitosan-copper hydrogel of the invention and/or prepared by any indicated method for producing a chitosan-copper hydrogel of the invention, for use as a medicine for the treatment of diseases and/or inflammations caused by bacteria, fungi, viruses, protozoa, preferably against bacteria of the genus Escherichia, Pseudomonas, Staphylococcus, Enterococcus, Acinetobacter, Proteus, fungi of the genus Candida, retroviruses, lentiviruses.

The invention also relates to a cosmetic or care composition for cosmetic, care and hygiene applications in humans and/or animals, which contains a chitosan-copper hydrogel of the invention and/or prepared by any indicated method for producing a chitosan-copper hydrogel of the invention, wherein the composition additionally contains at least one carrier approved for cosmetic, care and hygiene applications in humans and/or animals and is intended for external use. The cosmetic or care composition is preferably in the form of a liquid, emulsion, gel, cream, spray, hand washing gel, spray, wet wipe.

The invention also relates to a composition which contains a chitosan-copper hydrogel of the invention and/or prepared by any indicated method for producing a chitosan-copper hydrogel of the invention, for external use as a biocide, antiseptic, antibacterial agent, antiviral agent, fungicide, surface disinfectant, wherein, preferably, the composition is used against bacteria of the genus Escherichia, Pseudomonas, Staphylococcus, Enterococcus, Acinetobacter, Proteus, fungi of the genus Candida, retroviruses, lentiviruses, protozoa, wherein the composition is preferably in the form of a liquid, emulsion, gel, spray, lotion, wet wipe, paper towel, handkerchief, hygiene material.

The invention also relates to a veterinary composition which contains a chitosan-copper hydrogel of the invention and/or prepared by any indicated method for producing a chitosan-copper hydrogel of the invention, wherein the composition additionally comprises at least one carrier approved for veterinary use, wherein the composition is preferably in the form of a liquid, emulsion, gel, spray, foam, spray, liquid, cleansing gel, wet wipe or dressing, preferably the dressing is in the form of a patch with hydrogel, gauze with the composition applied, hydrocolloid dressing, hydrofibrous dressing, bandage with the composition applied.

The invention also relates to a veterinary composition which contains a chitosan-copper hydrogel of the invention and/or prepared by any indicated method for producing a chitosan-copper hydrogel of the invention for use as a medicine for the treatment of diseases and/or inflammations caused by bacteria, fungi, viruses, protozoa, preferably against bacteria of the genus Escherichia, Pseudomonas, Staphylococcus, Enterococcus, Acinetobacter, Proteus, fungi of the genus Candida, retroviruses, lentiviruses.

The invention also relates to the use of a chitosan-copper hydrogel of the invention and/or prepared by any indicated method for producing a chitosan-copper hydrogel of the invention, as an externally applied biocidal agent, antiseptic agent, antibacterial agent, bacteriostatic agent, antiviral agent, fungicide, agent for covering, soaking and disinfecting surfaces, preferably against bacteria of the genus Escherichia, Pseudomonas, Staphylococcus, Enterococcus, Acinetobacter, Proteus, fungi of the genus Candida, retroviruses, lentiviruses, protists.

In a preferred embodiment, the agent is used in health care to cover and soak the surfaces of fabrics, tools and devices used in medicine, veterinary medicine and diagnostics, and other surfaces, in particular hospital and laboratory surfaces, surfaces in public places, medical and veterinary clinics, handrails, door handles, toilets, taps, sanitary facilities, floors, walls.

In a preferred embodiment, the agent is used in the textile industry for covering fabrics and non-woven fabrics, preferably for covering medical fabrics and non-woven fabrics, preferably for covering fabrics and non-woven fabrics used to produce filters, air filters, medical textiles, gauze, dressings, bandages, masks protective clothing, medical gowns, protective suits, hospital mattresses, quilts, pillows, bedding, hospital bedding.

In a preferred embodiment, the agent is used in the cosmetics industry, preferably as an additive to cosmetics improving their microbiological quality, preferably as an additive to liquids, foams, gels, creams, milks, lotions.

In a preferred embodiment, the agent is used as a biocidal agent, antiseptic agent, antibacterial agent, bacteriostatic agent, antiviral agent, fungicide in the form of a liquid, emulsion, gel, foam, spray liquid, lotion, wet wipe or dressing, is preferably used as a bacteriostatic or bactericidal agent in the form of a plaster with a dressing, a patch with a hydrogel, gauze with the composition applied, a dressing, a bandage with the composition applied.

The invention also relates to the use of a chitosan-copper hydrogel of the invention and/or prepared by any indicated method for producing a chitosan-copper hydrogel of the invention. as an agent for purifying fluids from biological contaminants in the form of bacteria, fungi, viruses, protozoa by aggregation and inhibiting the growth of bacteria, fungi, viruses, protozoa on chitosan-copper hydrogel and sedimentation, preferably the purified fluids are biologically polluted waters, preferably waters in water treatment plants.

The invention also relates to a method for purifying aqueous solutions from microbiological contaminants in the form of bacteria, fungi, viruses, protozoa, in which a chitosan-copper hydrogel of the invention and/or produced by any indicated method for producing a chitosan-copper hydrogel of the invention is added to the contaminated aqueous solution; it is expected, preferably with stirring, for the formation of aggregates of microorganisms on the chitosan-copper hydrogel, the produced aggregates are isolated from the purified aqueous solution, preferably isolated by sedimentation.

Therefore, the invention is based on a chitosan-copper hydrogel composed of chitosan chains and cupric ions, which is stable in conditions of approximately neutral pH (in the range of pH 6-8, most preferably around pH 7 which itself has the ability to coat solid surfaces, preferably negatively charged, preferably hydrophilic or with low hydrophobicity (plastics, metals, glass and silica) and to soak porous and fibrous materials (for example: cellulose paper and tissue, glass, cotton, linen, wool, silk fabric and non-woven fabrics). Unexpectedly, it also covers polystyrene, which is not hydrophilic. The chitosan-copper hydrogel of the invention has a strong ability to immobilize, inhibit the growth and kill bacteria, viruses, yeasts and protozoa both in solution and on the surface coated or soaked with it. The chitosan-copper hydrogel of the invention has a much stronger biocidal effect than either of these substances alone, and it shows synergy of action and not the effect of simple addition.

According to the meaning used in the present description, the degree of chitosan deacetylation should be understood as the percentage of deacetylation to obtain reactive chitosan amino groups, where a 50% deacetylation degree means that 50% of chitosan units contain reactive amino groups, and 100% chitosan deacetylation means, respectively, that 100% of chitosan units contain reactive amino groups. By a mer of chitosan it should be understood a single glucopyranose ring, connected to other glucopyranose rings by O-glycosidic bonds, containing an acetamide group, as in chitin, before deacetylation, or an amino group, as in chitosan, after chitin deacetylation. By the degree of saturation of chitosan with copper(II) ions it should be understood the proportion of amino groups bound to copper(II) ions by a coordination bond to the total amino groups in chitosan, expressed in percentage values, wherein a 50% saturation degree means that 50% of the amino groups are bound to copper(II) ions, and a 100% saturation degree means, respectively, that 100% of the amino groups are bound to copper(II) ions.

Therefore, the degree of saturation of deacetylated chitosan with copper ions in less than 100% is determined based on the actual content of amino groups in this chitosan

### DETAILED DESCRIPTION OF THE INVENTION

In the method of obtaining the chitosan-copper hydrogel according to the invention a commercially available chitosan, characterized by the presence of reactive amino and hydroxyl groups, and an appropriate divalent copper salt are used.

In the method of obtaining the chitosan-copper hydrogel of the invention, a chitosan solution with a molar concentration ranging from 0.05 mM to 300 mM is used. Whereby 1% w/v chitosan corresponds to a molar concentration of 60 mM per each mer, and therefore the above molar concentrations correspond to w/v concentrations of 0.0008 to 5% w/v, preferably 15 to 180 mM (0.25 to 3% w/v), and a water-soluble organic or inorganic acid, such as acetic, lactic, formic, or other carboxylic acid, or a dilute inorganic acid, for example hydrochloric acid. Hereinafter, in the description and Examples, the symbol % when determining the concentration of chitosan or acid means weight-volume %, and when determining the degree of saturation of amino groups of chitosan with cupric ions and the degree of chitosan deacetylation it means mol %. In one of the production methods, after dissolving chitosan in dilute acid, preferably acetic acid, it should be dialyzed in distilled water, resulting in a chitosan solution separated from the acid and thus neutralized to pH about 7±1. A concentrated solution of inorganic or organic divalent copper salt, for example copper(II) chloride or acetate, is gradually added to the chitosan solution prepared in this way. The addition of the copper(II) salt solution should be accompanied by vigorous stirring, the aim of which is to evenly distribute the copper(II) in the structure of the unexpectedly formed hydrogel. An example of a possible manufacturing procedure is described in Example 1 pt A). Unexpectedly, the addition of excess copper(II) ions above the predicted molar ratio of copper(II) ions to chitosan monomers caused the hydrogel to disintegrate (FIG. 2A.J compared to FIG. 2A.I).

Example 1 pt B describes the method of creating a chitosan-copper hydrogel using chitosan with an 80% degree of deacetylation, unlike the one used in Example 1 pt A) chitosan with a degree of deacetylation close to 100%. As shown in FIG. 2B, a hydrogel was obtained with properties similar to the hydrogels obtained using the method according to Example 1 pt A). The preliminary tests carried out indicate that the minimum degree of deacetylation enabling the creation of the hydrogel according to the invention is 20%.

Alternatively, in other hydrogel production methods, as shown in Example 2, the chitosan-copper hydrogel is produced directly on the surface to be covered (object surface). Then, in one of such methods, the dialysis step can be skipped and, after adding copper(II) salt, the resulting hydrogel can be neutralized with a base, for example NaOH. The chitosan-copper hydrogel obtained in this way contains significant amounts of salts, which, however, does not affect its basic properties. This salt can be removed from the hydrogel by any method known in the art, for example by dialysis, rinsing.

Methods of producing a hydrogel directly on the covered surface in situ are advantageous in many applications, especially those described below, for example when there is a need to saturate a porous material with a high viscosity chitosan-copper hydrogel. It is then preferable to initially saturate such material with a lower viscosity chitosan solution, and then with a copper(II) solution of selected concentration, and then raise the pH to obtain the desired high-viscosity hydrogel in situ.

Alternatively, the viscosity of the produced hydrogel (e.g. obtained by the method according to Example 1, pt A) can be lowered by heating the chitosan-copper hydrogel to a temperature of 40-100° C., as described in Example 12.

In Example 3, pt A) the biocidal activity of the chitosan-copper hydrogel against Escherichia coli and Pseudomonas aeruginosa bacteria grown in liquid medium is disclosed. Unexpectedly, the hydrogel according to the invention retained its biocidal activity against these bacteria even after very significant dilution, up to approx. 50 M (0.0008%) against E. coli and up to approx. 100 M (0.0016%) against P. aeruginosa. In both cases, it was advantageous to use partial, rather than complete, saturation of amino groups of chitosan with copper(II) ions (Tab. 1).

In Example 3, pt B) the biocidal activity of the chitosan-copper hydrogel against the yeast Candida albicans is disclosed.

In Example 3, pt C) the biocidal activity of the chitosan-copper hydrogel against protozoa/protists Amoeba proteus is disclosed.

The results disclosed in Example 4 indicate that the biocidal mechanism of the hydrogel according to the invention includes the immobilization of microorganisms by the hydrogel, and therefore also the mechanical prevention of cell division or other forms of reproduction. In this way, the action of the chitosan-copper hydrogel, unlike the action of antibiotics, hinders the formation of mutations that could lead to the development of resistance to the action of the hydrogel.

In Example 5, the binding of pathogens by a film formed by drying the chitosan-copper hydrogel was demonstrated.

In Example 6, the biocidal activity of a film created by drying a chitosan-copper hydrogel layer under sterile conditions against E. coli and P. aeruginosa bacteria seeded on the film surface was demonstrated. For this purpose, a colony forming unit (CFU) test was used after exposing a bacterial culture to a surface covered with a hydrogel film for 1 hour. The results, illustrated in FIG. 11 for E. coli and FIG. 12 for P. aeruginosa, demonstrate a very strong biocidal effect of the hydrogels according to the invention, leading to the complete elimination of bacterial colonies. Contrary to tests in liquid culture, no weakening of the biocidal activity of hydrogels completely saturated with copper(II) ions was observed for films compared to partial saturation.

Preliminary research shows that the chitosan-copper hydrogel also has biocidal properties against bacteria of the genus Staphylococcus, Enterococcus, Acinetobacter, Proteus, fungi, especially of the genus Candida, single-cell parasites, especially protozoa, and viruses, including retroviruses, especially lentiviruses. Diseases and/or inflammation caused by bacteria of the genus Escherichia, Pseudomonas, Staphylococcus, Enterococcus, Acinetobacter, Proteus, fungi of the genus Candida, retroviruses, lentiviruses, for which chitosan-copper hydrogel can be used to treat, alleviate and/or prevent, include in particular diseases of the skin and mucous membranes, e.g. mastitis/mastitis in cows, skin infections caused by Pseudomonas, Staphylococcus.

In Example 7 the dependence of the viscosity of the hydrogel according to the invention on the degree of saturation of the amino groups of chitosan with copper(II) ions was described. For less concentrated hydrogels containing 15 mM (0.25%) of chitosan, the material retained fluidity comparable to the value for pure chitosan up to 50% saturation with copper(II) ions. However, for more concentrated chitosan solutions, the hydrogel form was also obtained in samples containing only 20% of saturation with copper(II) ions (and all samples with higher saturation) for 30 mM (0.5%) of chitosan, as well as 10% (and all samples with higher saturation) for 60 mM (1%) of chitosan. It has been shown that the chitosan-copper hydrogel according to the invention liquefies when heated.

As shown in Example 8, it unexpectedly turned out that in the chitosan-copper hydrogel according to the invention, one copper(II) ion simultaneously binds to only one amino group of chitosan (1:1 stoichiometry) with a saturation degree ranging from 5 to 100%. This is an unexpected behavior and opposite to that of glucosamine, which at neutral pH and an excess of glucosamine forms complexes in which the copper(II) ion binds two amino groups at the same time (2:1 stoichiometry, Micera et al. Inorg. Chim. Acta 107, 45-48, 1985). This means that the chitosan-copper hydrogel according to the invention creates a structure different from that known for glucosamine, otherwise, when the hydrogel was saturated with copper(II) ions up to 50%, complexes with a stoichiometry of 2:1 would be formed, and at higher saturation values a mixture of 2:1 and 1:1 stoichiometry would be observed. Moreover, unexpectedly, excess copper(II) ions bind to water molecules or, to a lesser extent, sugar hydroxyl groups, but without their deprotonation, as evidenced by the shift of the absorption band towards longer wavelengths, to the spectrum characteristic of copper(II) aqua-ion. Deprotonation of hydroxyl groups or water molecules by the copper(II) ion would cause the opposite effect, shifting the band to shorter wavelengths, because the maximum absorption of copper(II) hydroxide in solution is at 690 nm (R. Kotuniak, doctoral thesis, IBB PAN 2022). This means that in the hydrogel according to the invention, despite the high water content, and in Example 8 over 99.5% by weight, the environment of copper(II) ions is unexpectedly fundamentally different from that known for an aqueous solution.

It follows that the rules for forming copper(II) complexes with glucosamine, described in Micera et al. Inorg. Chim. Acta 107, 45-48, 1985, do not apply to the binding of copper(II) ions to chitosan, because in the presence of glucosamine monomer, the Cu²⁺ ion at pH 7 had the ability to connect two amino sugar units through Cu—N bonds. The shift of the absorption band towards longer wavelengths when the content of Cu²⁺ ions in the hydrogel is above 100% in relation to the amino groups indicates that the excess copper(II) ions are in the form of aqua-ions. These excess aqua-ions break the structure of the hydrogel, which results in a decrease in its viscosity and visible delamination of its structure. However, they still interact with chitosan chains, as evidenced by the lack of formation of copper(II) hydroxide. Primarily, however, this is done through hydrogen bonds, as evidenced by the absorption spectrum of such a hydrogel, characteristic of copper(II) aqua-ion. Taken together, the test results presented in Examples 7 and 8 allow us to propose the structure of the chitosan-copper hydrogel according to the invention, shown in FIG. 1B1 and FIG. 1B2. In this structure, the hydrogel is formed by cross-linking of chitosan chains by copper(II) ions via coordination bonds with amino groups and hydrogen bonds between the coordination water molecules of copper(II) ions and the hydroxyl groups of chitosan. Counterions from the copper(II) salt used, e.g. chlorides, interact with copper(II) ions ionically, outer-sphere, without affecting the properties of the hydrogel. Therefore, unexpectedly, at pH 7±1 the copper(II) ion forms interactions through one amino group, anchoring the ion to the chitosan chain and water molecules and hydroxyl groups, which allows cross-linking of these chains to form a hydrogel. As shown in Example 9, cobalt(II), nickel(II) and zinc(II) ions do not have this ability, only the copper(II) ion has it.

The results presented in Example 8 confirm that for the chitosan used with a deacetylation degree close to 100%, the theoretical saturation of amino groups with copper(II) ions, assumed in the previous examples, corresponds to the actual saturation of these groups, which takes place by the formation of a single Cu(II)-N bond. However, from Example 1 pt B), as well as preliminary tests, it follows that batches of chitosan from different suppliers have different contents of amino groups, which results from different degrees of deacetylation of the starting material, chitin. The results of Example 8 indicate the validity of knowing and/or controlling the content of amino groups in chitosan before preparing a chitosan-copper hydrogel for applications according to the invention, and at the same time Example 8 provides the procedure for implementing such control, for example, by spectrophotometric titration of a chitosan solution with a copper(II) salt solution and analysis of the position of the maximum of the absorption band of the copper(II) ion. Alternative methods of monitoring the deacetylation/content of reactive amino groups of chitosan are possible, provided they allow for a clear determination of the amount of copper ions bound to the amino groups of chitosan. These include, e.g., circular dichroism, electron paramagnetic resonance, infrared spectroscopy, etc.

Comparison of the data from Example 8 with the biocidal properties of the chitosan-copper hydrogels of the invention, described in Examples 3-6, and with the viscosity data of the hydrogels obtained for 15, 30 and 60 mM (0.25%, 0.5% and 1%, respectively) chitosan solution, described in Example 7, indicates that partial, rather than complete saturation of the amino groups of the hydrogel according to the invention with copper(II) ions is particularly advantageous for the biocidal properties in the solution and related to their applications. For hydrogels with saturation in the range of 50-70%, the biocidal effect is particularly strong and remains significant even at very significant dilution, even more than a thousand times of the initial hydrogel. In applications related to the biocidal activity of the film formed by the chitosan-copper hydrogel of the invention and the surfaces covered with a film made of this hydrogel, a high, especially complete saturation of the amino groups of the hydrogel with copper(II) ions is beneficial, while the biocidal effect is also achieved for partial saturations. The high viscosity of the hydrogel at such saturations may be beneficial for many applications, for example those related to surface covering. As demonstrated in Example 7 (FIG. 13), this is preferably achieved by using a hydrogel with a chitosan concentration of at least 30 mM (0.5%). Therefore, for the experiments from Examples 8-12, a preferred 30 mM (0.5%) chitosan-copper hydrogel with 50% saturation of its amino groups with copper(II) ions was selected for further research.

In Examples 10 to 12 methods of coating of a wide range of solid and porous surfaces and soaking the porous surfaces with the chitosan-copper hydrogel of the invention were described. The hydrogel creates compact coverings that adhere closely to the substrate, especially on hydrophilic surfaces capable of forming hydrogen bonds, such as those made of cellulose and sodium glass. Unexpectedly, however, it forms analogous coverings on surfaces that do not have this ability, such as aluminum and polystyrene surfaces (Example 10, FIG. 15). The ready-to-use chitosan-copper hydrogel prepared (e.g. by the method according to Example 1, pt A)) has the ability to soak porous materials, such as cellulose paper, glass and cotton fabric (Example 11, FIG. 16), as well as linen, woolen and silk fabrics. The same applies to the chitosan-copper hydrogel obtained by the in situ preparation method (as in Example 2), as demonstrated in Example 12 for cotton fabric (FIG. 17). Covering porous materials with the hydrogel of the invention by soaking/applying or producing hydrogels in situ, i.e. directly on the covered surfaces, gives a lasting effect. The hydrogel remained on the covered surfaces after being soaked for 24 hours in a solution of a strong ionic detergent (1% SDS), as demonstrated in Example 13 (FIG. 18). This means that covered materials, i.e. coated or soaked with hydrogel according to the invention, retain their properties in the process of washing and cleaning with detergents.

As shown in Example 14, the hydrogel of the invention retains its properties after long-term (at least 24 months) storage in a dried form at room temperature. Reconstitution requires adding an appropriate amount of water and briefly boiling and cooling the suspension. The hydrogel can be stored for a long time (at least 24 months) in a undried form, in a closed container at the temperature of 4° C., i.e. the temperature of a typical refrigerator. For pouring, soaking, covering and other applications where it would be beneficial to reduce its viscosity, this can be achieved by heating it to a higher temperature. After cooling, the hydrogel restores all its physico-chemical and biological properties.

Copper ions are known in the art for their ability to produce reactive oxygen species (ROS) through the Fenton and Haber-Weiss cycle of chemical reactions involving the Cu(II) and Cu(I) redox states. This activity underlies the biocidal properties of copper and its compounds (Wang F et al. Curr Med. Chem 17, 2685-2698, 2010). It is particularly high for weakly bound Cu(II) ions, because the stronger binding of the Cu(II) ion shifts the Cu(II)/Cu(I) redox balance towards Cu(II), thereby reducing and, in extreme cases, completely inhibiting the formation of RFT (Wiloch M Z et al. J Electrochem Soc 163, G196-G199, 2016). In special cases of biological or synthetic molecules containing oxygen and nitrogen atoms to which the Cu(II) ion binds, a larger number of bound nitrogen atoms results in a stronger binding of the Cu(II) ion, but lower redox activity. In oxidation-reduction enzymes whose activity is based on the Cu(II)/Cu(I) pair, this limitation is removed by the specific structural properties of proteins, which are difficult to reproduce in complexes of small molecules and organic polymers (Villafranca J J. et al., in Bioinorganic Chemistry of Copper. Springer, Dordrecht. https://doi.org/10.1007/978-94-011-6875-5_35). Unexpectedly, in the chitosan-copper hydrogel of the invention, the strong binding of Cu(II) ions, which is evidenced by the stability of the hydrogel and the preservation of its biocidal properties over long periods of storage (Example 14), did not lead to a limitation of the oxidizing capacity of cupric ions, thanks to the fact that they are bound to the chitosan chains via only one nitrogen atom (as demonstrated in Example 8). This was possible thanks to the specific structure of the hydrogel of the invention, in which the strong binding of Cu(II) ions was achieved thanks to their immobilization in a network of hydrogen bonds between water molecules bound to the Cu(II) ion and chitosan chains. This method of binding, based on a large number of individually weak chemical bonds, does not disturb the redox properties of the Cu(II) ion, which are similar to those known in the field of weakly bonded copper ions, for example Cu(II) aqua-ion.

In Examples 15 to 17 it was demonstrated that the chitosan-copper hydrogel has strong oxidizing abilities without the need to add chemical oxidants to it. Example 15 shows that purified HyPer7 protein, which is a marker for reactive oxygen species, is oxidized by the hydrogel. In Example 16 it was shown that the HyPer7 protein is also oxidized by the hydrogel inside the bacterial cell. In Example 17 it was shown that the oxidative capacity of the hydrogel also leads to the oxidation of lipids in bacterial cells, which leads to their peroxidation.

### BRIEF DESCRIPTION OF FIGURES OF THE DRAWING

The invention in the embodiments is explained in more detail with reference to the non-limiting figures of the drawing, in which:

FIG. 1 shows structures A1-A2 according to the prior art. A1: structure of copper(II) complexes with glucosamine according to Micera et al. 1985. The structure elements in square brackets appear in particular forms of the complex depending on the pH and the molar ratio of glucosamine to copper(II). At neutral pH, with an excess of glucosamine over copper(II), a complex in which the cupric ion is bound to two nitrogen atoms is preferred; A2: structure of the copper(II) complex in the chitosan-copper-ammonia hydrogel according to Li et al. 2019. In this structure, the copper(II) ion is bonded to two nitrogen atoms. B1 shows the basic subunits that make up the chitosan-copper hydrogel: basic subunit I (formula I)—which consists of non-deacetylated glucosamine subunits of chitosan, basic subunit II (formula II)—which consists of deacetylated glucosamine subunits of chitosan with reactive amino groups of chitosan, unsaturated with copper(II) ions, basic subunit III (formula III)—which consists of deacetylated glucosamine subunits of chitosan with reactive amino groups of chitosan, saturated with copper(II) ions. Formula III also illustrates the binding method of a single copper(II) ion to the glucosamine subunit of chitosan in such a subunit, forming a chitosan-copper hydrogel; B2 shows the illustrated structure of the chitosan-copper hydrogel according to the invention. In B2 the situation for 100% saturation of the hydrogel with cupric ions, according to formula III is shown. At lower saturation, some glucosamine residues do not form bonds with cupric ions, according to formula II. In this structure, the hydrogel is formed by the cross-linking of chitosan chain ions by copper(II) ions via coordination bonds with amino groups and hydrogen bonds between the coordination water molecules of copper(II) ions and the hydroxyl groups of chitosan. In FIG. 1B2 the structure of the chitosan-copper hydrogel is shown, with the basic units of the chitosan-copper hydrogel with 100% saturation with copper(II) ions, produced through a network of hydrogen bonds from the combination of six basic units (of formula III), which explains how the basic units are connected together. Solid lines represent covalent and coordination bonds, and dashed lines represent key hydrogen bonds. Negative ions interact with copper(II) ions in the outer sphere mode, chloride ions are shown in FIG. 1B2, but they may be other negative ions, depending on the copper(II) salt used. The symbol R¹ stands for chains composed of subunits according to formulas I, II and III, depending on the composition of the hydrogel. The chain length depends on the length of the chitosan chains.

FIG. 2 shows the formation of chitosan-copper hydrogel, depending on the saturation of amino groups with copper(II) ions. A) hydrogel obtained by the method from Example 1, pt A. Control: 60 mM (1%) of chitosan solution (A) and, respectively, chitosan-copper hydrogel with saturation of amino groups with copper(II) ions in the following degrees of saturation of amino groups: 10% (B), 20% (C), 30% (D), 40% (E), 50% (F), 70% (G), 80% (H), 100% (I), 500% (J) (Example 1 pt A)). B) chitosan-copper hydrogel with 100% saturation of amino groups with copper(II) ions, obtained using the method from Example 1 pt B).

FIG. 3 shows the growth curves of Candida albicans yeast in the presence of subsequent dilutions of chitosan-copper hydrogel with a saturation of amino groups of 100% (Example 3B).

FIG. 4 shows a microscopic image of an Amoeba proteus culture before (A) and after 1 min. (B) after adding chitosan-copper hydrogel to the culture at a concentration of 1 mM of chitosan and 100% saturation with Cu(II) ions (Example 3C).

FIG. 5.1 shows photos of liquid bacterial cultures in the stationary phase (Example 3); respectively: (A) E. coli without hydrogel—visible light, 25× magnification; (B) E. coli with chitosan-copper hydrogel at a concentration of 3 mM (0.05%) and 50% saturation of amino groups 10 with copper(II) ions, visible light, magnification 25×; (C) E. coli without hydrogel, Nomarski contrast, 400× magnification; (D) E. coli without hydrogel, Nomarski contrast, 400× magnification, Hoechst 33258 staining for DNA visualization; (E) E. coli in hydrogel as in (B), Nomarski contrast, magnification 400×; (F) E. coli in hydrogel as in B, Nomarski contrast, 400× magnification, staining with Hoechst 33258 dye for DNA visualization (Example 4).

FIG. 5.2 shows the immobilization of bacteria by chitosan-copper hydrogel. For visualization, Escherichia coli bacteria were used, fluorescing in blue light by overexpressing the GFP protein. (A) control (Escherichia coli bacteria in the medium) in visible light; (B) blue light control (excitation 488 nm, emission 525 nm); (C) bacteria in medium containing 1 mM of chitosan solution, under visible light; (D) bacteria in a medium containing 1 mM of chitosan solution under blue light (excitation 488 nm, emission 525 nm); (E) bacteria in a medium containing 1 mM of chitosan solution with 50% saturation with copper(II) ions, under visible light; (F) bacteria in medium containing 1 mM of chitosan solution with 50% saturation with copper(II) ions, under blue light (excitation 488 nm, emission 525 nm); (G) bacteria in a medium containing 1 mM of chitosan solution with 100% saturation with copper(II) ions, under visible light; (H) bacteria in a medium containing 1 mM of chitosan solution with 100% saturation with copper(II) ions, under blue light (excitation 488 nm, emission 525 nm). Fluorescence (emission at 525 nm) recorded by a monochromatic camera is seen as bright light (Example 4).

FIG. 6 shows the immobilization of Candida albicans yeast by a chitosan-copper hydrogel with a concentration of 12 mM (0.2%). In the presence of hydrogel, the yeast cells Candida albicans are immobilized, which is confirmed by observing the formation of small aggregates (B), compared to the medium without hydrogel (A). (Example 4).

FIG. 7 shows the immobilization of bacteria by a film formed on a glass surface by a dried chitosan-copper hydrogel. Escherichia coli bacteria were used, fluorescing due to the overexpression of the GFP protein, visualized in blue light (excitation 488 nm, emission 525 nm). White dots represent single bacterial cells. (A) image after 10 min of incubation; (B) image after 10 min of incubation and three washes with saline solution (Example 5).

FIG. 8 shows the immobilization of the yeast Candida albicans by a film formed on a glass surface by a dried chitosan-copper hydrogel. The dots in the microscopic image represent individual yeast cells. (A) image after 10 min of incubation; (B) image after 10 min of incubation and three washes with saline solution (Example 5).

FIG. 9 shows the effect of the film formed by the hydrogel over time. Amoeba on a glass surface (A, C, E, G). Amoeba on the surface of a film formed by a hydrogel (B, D, F, H). Pictures were taken every 30 seconds (Example 5).

FIG. 10 shows the immobilization of Paramecium tetraurelia protists by a film formed on a glass surface by a dried chitosan-copper hydrogel. Photos A and B were taken in visible light at an interval of 1 second. The arrows indicate Paramecium cells that moved after 1 second. Paramecium cells that did not change their position were marked with asterisks. Photo C shows an automatic comparison of both images in ImageJ, highlighting the differences (Example 5).

FIG. 11 shows a graph showing the ability to inhibit the formation of E. coli colonies by a film formed by a chitosan-copper hydrogel covering a polystyrene surface (Example 6).

FIG. 12 shows a graph showing the ability to inhibit the formation of P. aeruginosa colonies by a film created by a chitosan-copper hydrogel covering a polystyrene surface (Example 6).

FIG. 13 shows photos of chitosan-copper hydrogels, illustrating the dependence of viscosity on the degree of saturation of amino groups of chitosan with copper(II) ions, taken after turning the test tubes upside down and waiting 1 minute. Top row: 30 mM (0.5%) chitosan-copper hydrogel with saturation of amino groups with copper(II) ions equal to 10% (A), 20% (B), 40% (C), 80% (D), 100% (E), 500% (F). Bottom row: 60 mM (1%) chitosan-copper hydrogel with saturation of amino groups with copper(II) ions 10% (G), 20% (H), 40% (I), 80% (J), 100%, 500% (L).

FIG. 14 shows the dependence of the absorption band on the degree of saturation of the amino groups in chitosan by copper(II) ions (Example 8). Left panel: absorption spectra of the hydrogel depending on the content of copper(II) ions, the solid arrow indicates the direction of spectrum changes as the content of copper(II) ions in the hydrogel increases. Asterisks mark the location of the absorption maximum. The dashed arrow indicates the range and direction of changes in the position of the absorption maximum. Right panel: absorbance values determined by averaging in the range of 700-800 nm, and then averaging over four measurement series.

FIG. 15 shows photos of solid surfaces covered by the hydrogel according to the invention: polystyrene (A); aluminum (B); soda glass (C) before (1) and after covering (2), respectively; silica (D) before (1) and after covering (2) respectively (Example 10).

FIG. 16 shows the coverage and saturation of porous materials by the hydrogel according to the invention: filter paper (A); glass filter (B); cotton fabric (C) (Example 11).

FIG. 17 shows photos of chitosan-copper hydrogel deposited on Whatman 3MM filter paper. Blotting paper with embedded hydrogel before rinsing (A); the same tissue paper after 5 minutes of rinsing with running water (B) (Example 13).

FIG. 18 shows photos from testing the durability of chitosan-copper hydrogel deposition during rinsing with an ionic detergent: Whatman 3MM tissue paper soaked in hydrogel (A); Whatman 3MM tissue paper with (A) after 24 h rinsing in 1% SDS (B); Whatman GF/A glass filter soaked in hydrogel (C); filter from (C) after 24 h of rinsing in 1% SDS (D); cotton fabric soaked in hydrogel (E); fabric from (E) after 24 h of rinsing in 1% SDS (F) (Example 13) FIG. 19 shows the evolution over time of the oxidation level of the HyPer7 protein, measured as the proportion of fluorescence emission at 520 nm with excitation at 488 nm (oxidized protein) to fluorescence at 520 nm with excitation at 405 nm (reduced protein), in reaction to the chitosan-copper hydrogel of the invention and control reactions: 10 mM H₂O₂ (positive control, oxidized protein), and 10 mM dithiothreitol (DTT, negative control, reduced protein) (Example 15).

FIG. 20 shows the evolution over time of the oxidation level of the HyPer7 protein inside an 20 Escherichia coli cell, measured as the proportion of fluorescence emission at 520 nm with excitation at 488 nm (oxidized protein) to fluorescence at 520 nm with excitation at 405 nm (reduced protein), in reaction to the chitosan-copper hydrogel according to the invention and control reactions (Example 16).

FIG. 21 shows the effect of chitosan-copper hydrogel on the level of lipid peroxidation of 25 Escherichia coli bacteria (Example 17).

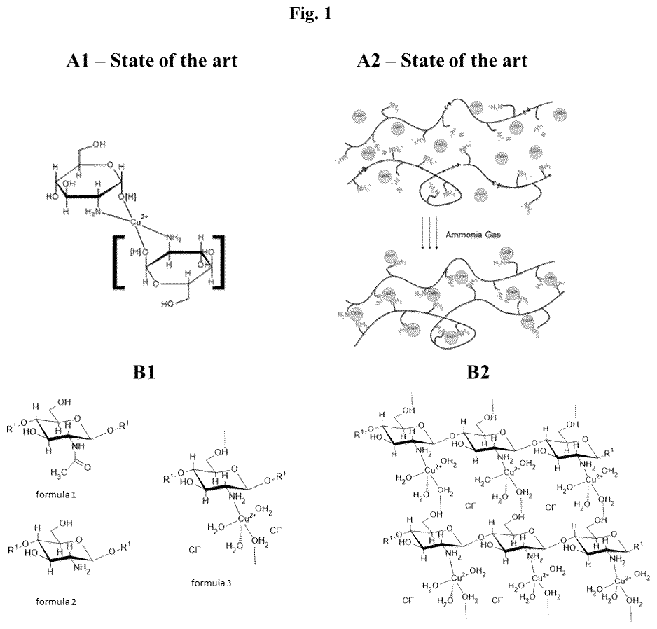

### METHODS OF CARRYING OUT THE INVENTION

Examples of embodiments of the invention are presented below. It should be noted that the examples below do not constitute a limitation of the invention, but represent its most advantageous implementation.

### EXAMPLES

### Example 1. Preparation of Ready-Made Chitosan-Copper Hydrogel

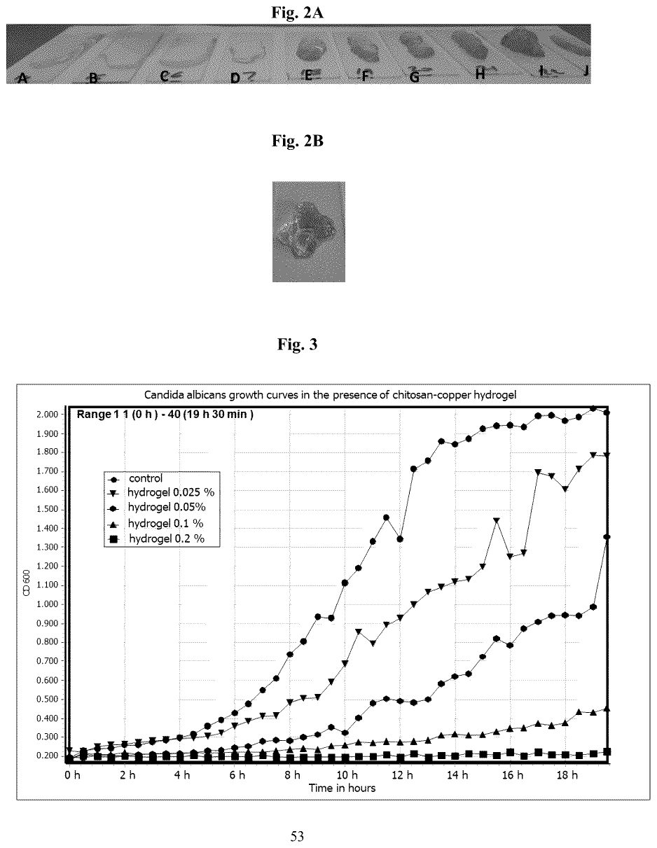

A) 12.4 mmol (2 g) of chitosan, (Sigma-Aldrich, catalog number 448869), was dissolved in 100 ml of an aqueous solution of 1% acetic acid at room temperature, obtaining a chitosan solution with a concentration of 120 mM based on chitosan monomer, i.e. 2 weight-volume % (dissolution in other organic and inorganic acids, for example formic or lactic acid, is acceptable and equivalent). The pH was adjusted to approximately 7 (pH range 6-8 is acceptable) by dialysis in distilled water. As a result of dialysis, the volume of chitosan doubled, which means that the concentration of the obtained chitosan solution at pH 7 is 60 mM (1%). To this solution, an aqueous solution of copper(II) chloride with a concentration of 1 M was gradually added, with vigorous stirring (it is permissible to use sulfate, nitrate, or other inorganic or organic salts of divalent copper), in an amount corresponding to the saturation of the amino groups of chitosan in a molar ratio of 5 to 100%. After chitosan binds Cu²⁺ ions, a blue hydrogel is formed, with a higher density and viscosity than the chitosan solution itself. A significant (e.g. five-fold) excess of copper(II) ions unexpectedly causes the structure of the hydrogel to collapse, which results in a loss of homogeneity of the substance. Chitosan-copper hydrogels with various degrees of saturation of amino groups with copper(II) ions were produced and their appearance and properties were examined. 2 ml of the obtained hydrogels and 30 mM (1%) of chitosan solution as a control were applied to coverslips, the appearance of which is shown in FIG. 2A. Hydrogels with similar properties, but lower concentration and proportionally lower density and viscosity can be obtained in an analogous way by dissolving smaller amounts of chitosan, for example 1 g, 0.5 g, 0.25 g in a volume of 100 ml, and hydrogels with similar properties but higher concentration and proportionally higher density and viscosity, dissolving increased amounts of chitosan, e.g. 2, 3, 4, 5, 10 g in a volume of 100 ml.

B) 12.4 mmol (2 g) of chitosan with a degree of deacetylation of 80%, (Suvidhinath Laboratories, Technical & Industrial Grade), was dissolved in 100 ml of an aqueous solution of 1% acetic acid at room temperature and dialyzed as in Example 1A to obtain a solution with a concentration of 30 mM (1%). To this solution, an aqueous solution of copper(II) chloride with a concentration of 1 M was gradually added, with vigorous stirring (it is permissible to use sulfate, nitrate, or other inorganic or organic salts of divalent copper), in an amount corresponding to the saturation of amino groups of chitosan in a molar ratio of 100%, which corresponds to the amount of Cu²⁺ ions used to create the hydrogel in Example 1 pt A), containing 80% saturation with Cu²⁺ ions. After chitosan binds Cu²⁺ ions, a blue hydrogel is formed, with a higher density and viscosity than the chitosan solution itself, shown in FIG. 2B.

### Example 2. Preparation of Chitosan-Copper Hydrogel In Situ

A) 12.4 mmol (2 g) of chitosan was dissolved in 100 ml of 1% acetic acid, as in Example 1 pt A), obtaining a solution with a concentration of 120 mM based on the chitosan monomer, i.e. 2 weight-volume % (dissolution in other organic and inorganic acids, e.g. formic or lactic acid, is acceptable). A solution of copper(II) chloride with a concentration of 1 M was added to the chitosan solution, with vigorous stirring (other inorganic or organic salts of divalent copper can be used), to a concentration of 60 mM, corresponding to 50% saturation of the amino groups with cupric ions (or other corresponding to the saturation of amino groups at the level of 5-100%). Then, a 0.1 M NaOH solution was added to the obtained mixture until pH 7 was obtained, which resulted in the appearance of a blue color of the hydrogel (it is possible to use another strong base or buffer, as long as they do not contain agents chelating copper ions). Hydrogels of lower concentration can also be produced, e.g. by dissolving 1 g, 0.5 g or 0.25 g of chitosan in 100 ml of acid.

B) Alternatively, the pH of the chitosan solution can be adjusted to pH 6-8 and then a copper salt solution with a concentration corresponding to the saturation of the amino groups at 5-100% can be added.

Therefore, in these embodiments, in order to produce a hydrogel in situ directly on a specific surface, e.g. on a solid material such as glass, fabric or non-woven fabric, such surface i) is first contacted with a mixture of a solution of chitosan in acid with a solution of inorganic or organic copper (II) salt of a selected concentration, e.g. by applying, spreading, spraying, soaking or immersing, then the surface is contacted with the alkali solution until the pH is approximately 7 and a hydrogel is formed (the appearance of a blue color of the hydrogel) or ii) first, the surface is contacted with a chitosan solution with a set pH of approximately 7, e.g. by applying, spreading, spraying, soaking or immersing, then the surface is contacted with a solution of inorganic or organic copper(II) salt of a selected concentration to form a hydrogel in situ on the surface.

The hydrogel obtained by the in situ production method according to Example 2 in production version A or in production version B has the same physicochemical and biological properties as the one obtained according to the method from Example 1 pt A), but the final concentration is twice as high because no dialysis is used. The only difference in the composition is the presence of acetic acid salt, which can, if necessary, be removed using methods known from the state of the art, e.g. rinsing, dialysis.

### Example 3. Biocidal Activity of Chitosan-Copper Hydrogel in Liquid Culture

A) In order to demonstrate the increased biocidal activity of the chitosan-copper hydrogel compared to the chitosan solution and the copper(II) salt solution of comparable concentration, bacteria were cultured in liquid medium in successive dilutions of the hydrogel. A 1% hydrogel prepared as in Example 1, pt A) with various levels of saturation of amino groups with copper(II) ions was diluted in a ratio of 1:10, obtaining a hydrogel concentration of 6 mM (0.1%). Then, subsequent 1:2 dilutions of such chitosan-copper hydrogel were used. Escherichia coli strain EC25922 and Pseudomonas aeruginosa strain PA01 were cultured in minimal medium. (doi:10.1101/pdb.rec12295 ColdSpring Harb Protoc 2010). The starter culture was diluted to OD=0.1 and grown with shaking in a 96-well plate, OD was measured every 30 min for 24 h. The volume of a single well was 100 μl. Culture was performed and OD was measured in a universal plate reader (FLUOstar Omega, BMG Labtech). The chitosan-copper hydrogel shows increased biocidal properties compared to chitosan or copper salts of the same concentration, which indicates the synergy effect of both of these biocidal factors interacting in the hydrogel. Example results are presented in Tab. 1. Low values mean inhibition of bacterial growth.

The data in Tab. 1 show that the chitosan-copper hydrogel has a bactericidal effect against bacteria in liquid culture, especially effectively when the amino groups are partially saturated with copper ions. The optimal saturation for E. coli is approximately 50%, and for P. aeruginosa 60-70%. Unexpectedly, the chitosan-copper hydrogel retains significant biocidal activity even in significant dilution, at a concentration of approx. 50 μM against E. coli and approx. 100 μM against P. aeruginosa.

Comparison of the results of the action of chitosan, copper(II) chloride and the hydrogel according to the invention for P. aeruginosa proves the synergy of the action of both hydrogel components. These bacteria are insensitive to the action of copper(II) ions in the medium, so the synergy is evidenced by an increase in the activity of the hydrogel towards chitosan alone at concentrations of 750 and 375 μM based on the chitosan monomer, for all tested degrees of saturation with copper(II) ions, and at concentrations of 187 and 94 μM, especially at 70% saturation. For E. coli, copper(II) ions no longer inhibit bacterial growth at concentrations of 100 μM and lower. This concentration range corresponds to all hydrogels presented in Tab. 1 with a chitosan concentration of 94 μM and lower. At a hydrogel concentration of 94 μM, significantly lower bacterial growth was observed for saturations with copper(II) ions in the range of 30-90%, and for a concentration of 47 μM in the range of 30-60%, which also demonstrates the synergy of the action of chitosan and copper(II) in the hydrogel for these concentration ranges of its components.

B). In order to demonstrate the biocidal activity of the chitosan-copper hydrogel against the yeast Candida albicans, the yeast was grown in complete minimal medium, in a culture containing successive dilutions of the hydrogel containing various levels of saturation of the amino groups with copper(II) ions (FIG. 3). The initial dilution of the starting hydrogel was 1.5, resulting in a 15 hydrogel concentration of 12 mM, based on amine groups (0.20%). Subsequent two-fold dilutions of such chitosan-copper hydrogel were then used. The starter culture was diluted to optical density (GD)=0.2 and grown with shaking in a 96-well plate at 30° C. OD(600) was measured every 30 min for 20 h using a universal plate reader (FLUOstar Omega, BMG Labtech). The volume of a single well was 100 μl. Hydrogel with a concentration of 12 mM (0.20%) completely blocked the growth of yeast in the tested culture.

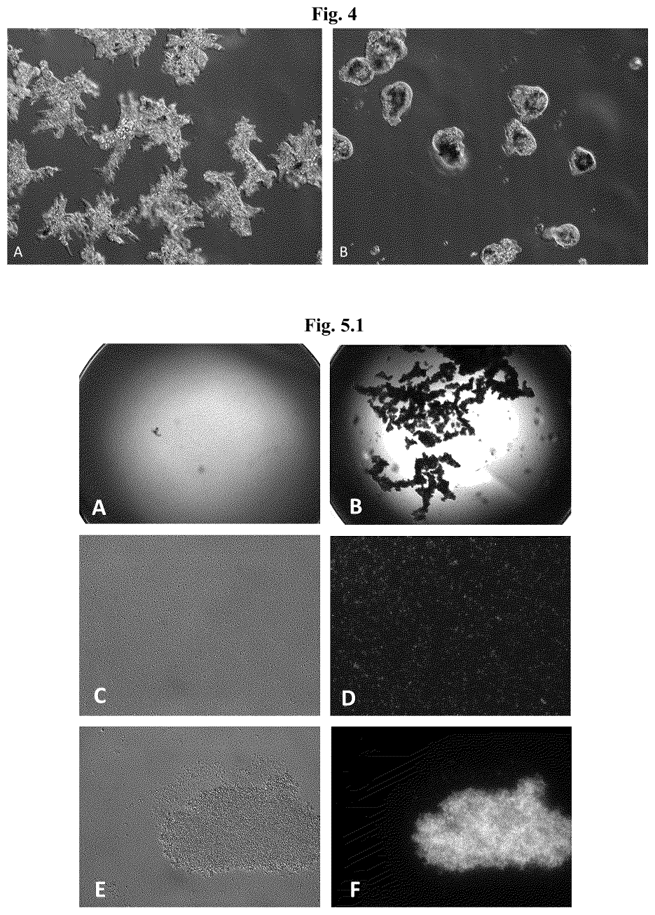

C). In order to demonstrate the biocidal activity of the chitosan-copper hydrogel against the protist Amoeba proteus, a chitosan-copper hydrogel saturated with Cu(II) ions to a concentration of 1 mM was added to the amoeba culture in liquid medium and microscopic observation was performed. (FIG. 4). After 1 min, all amoebae shrank and the movement of vesicles inside the cell stopped completely, indicating the death of the protist cell.

### Example 4. Binding of Pathogens by Chitosan-Copper Hydrogel in a Liquid

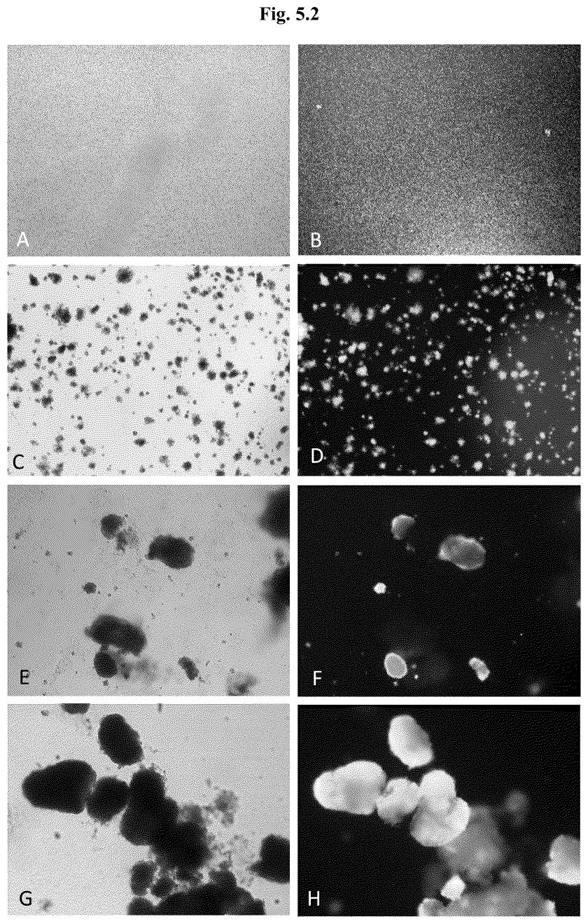

A.1). In order to demonstrate the mechanism of action of the chitosan-copper liquid hydrogel, E. coli strain EC25922 and P. aeruginosa strain PA01 were cultured in minimal medium in dilutions of the hydrogel as in Example 3, pt A). After 12 h, aggregates were observed in the culture grown in the presence of dissolved hydrogel. In order to demonstrate the presence of bacteria in the aggregates, the culture was stained with Hoechst 33258 dye, which stains DNA, and the preparation was observed under a fluorescence microscope equipped with Nomarski optics, at 365 nm excitation. As documented in FIG. 5.1, the hydrogel, even when significantly diluted, binds and immobilizes bacteria, causing them to form aggregates. These aggregates are visible to the naked eye (FIG. 5.1.B). Staining of the aggregates with the DNA-binding dye Hoechst 33258 shows that the aggregates consist of bacteria (FIG. 5.1.F). In the control without the addition of hydrogel, the bacteria are distributed evenly in the well of the plate (FIG. 5.1.D). Through aggregation, the hydrogel additionally inhibits the ability of bacteria to migrate and divide. It can therefore be used to protect against infections and to purify aqueous solutions from microorganisms.

A.2.) In order to demonstrate the mechanism of action of the biocidal activity of the chitosan-copper hydrogel according to the invention, E. coli bacteria overexpressing the fluorescent GFP protein were cultured in minimal medium. (doi:10.1101/pdb.rec12295 Cold Spring Harb Protoc 2010). The starter culture was diluted to OD=0.1 and grown with shaking in a 96-well plate, OD was measured every 30 min for 24 h. The volume of a single well was 100 μl. Culture was performed and OD was measured in a universal plate reader (FLUOstar Omega, BMG Labtech). The culture was carried out in the presence of chitosan-copper hydrogel with various degrees of hydrogel saturation with copper and in various dilutions. The GFP protein, produced inside bacteria, gives the bacteria fluorescent properties. When excited by blue light (excitation at 488 nm), bacteria fluoresce green (emission at 560 nm). FIG. 5.2 (A-H) shows microscopic images in visible light and blue light. In bacterial culture without the addition of hydrogel, single spots are visible, which correspond to single bacterial cells. In bacterial culture with the addition of 0.016% chitosan solution according to the invention (corresponding to the theoretical concentration of amino groups of chitosan, equal to 1 mM), bacterial cells are captured by chitosan chains and form fine aggregates. Chitosan-copper hydrogel with the same chitosan concentration and 50% saturation of amino groups with copper (II) ions produce significantly larger bacterial aggregates. Chitosan-copper hydrogel with the same chitosan concentration and 100% saturation of amino groups produces the largest observed aggregates. This means that the chitosan hydrogel itself has a moderate ability to immobilize bacteria, while the chitosan-copper hydrogel has a very high ability to immobilize bacteria, which is determined by the copper content in the hydrogel.

B). In order to demonstrate the ability of the chitosan-copper hydrogel to bind the yeast Candida albicans, the yeast was cultured in complete minimal medium containing successive dilutions of the hydrogel containing various levels of saturation of the amino groups with copper(II) ions. The initial dilution of the starting hydrogel was 1:5, resulting in a hydrogel concentration of 12 mM, based on amine groups (0.2%). Subsequent two-fold dilutions of such chitosan-copper hydrogel were then used. The starter culture was diluted to optical density (OD)=0.2 and grown with shaking in a 96-well plate at 30° C.

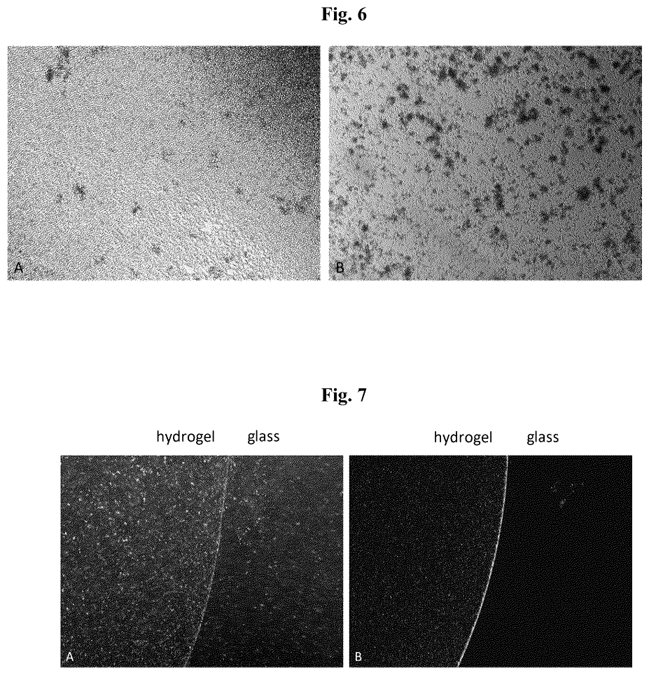

Hydrogel with a concentration of 12 mM (0.2%) partially binds Candida albicans yeast cells, creating fine aggregates (FIG. 6).

### Example 5. Binding of Pathogens by a Film Made of Chitosan-Copper Hydrogel

A). In order to demonstrate the ability of the chitosan-copper hydrogel deposited on the surface to bind bacteria, a glass microscopic coverslip was covered with a drop of hydrogel with a concentration of 10 mM and 100% saturation with copper ions and left to dry. Then, a culture of Escherichia coli overexpressing the GFP protein was placed on the border of the film formed by the hydrogel. Bacteria were visualized under blue light as in Example 4A.2. Bacterial cells are visible as dots. After 10 minutes of incubation and three washes with saline solution, the bacteria are visible only on the surface covered with hydrogel, but they disappeared from the glass surface. (FIG. 7). This means that the saline solution washed the bacteria from the glass surface, but it could not do so with the bacteria immobilized by the surface of the chitosan-copper hydrogel according to the invention.

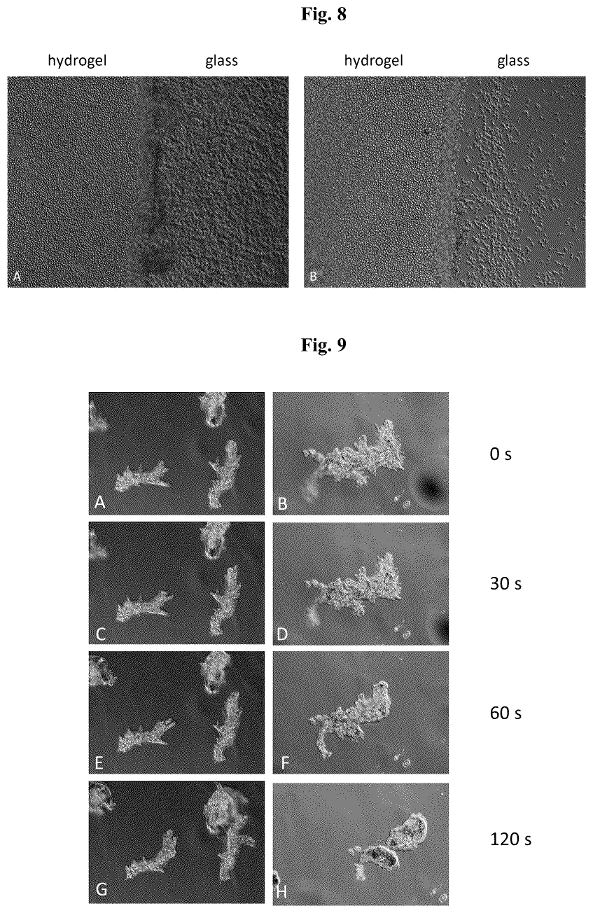

B). In order to demonstrate the ability of the chitosan-copper hydrogel deposited on the surface to bind the yeast Candida albicans, a glass microscopic coverslip was covered with a drop of hydrogel with a concentration of 10 mM and 100% saturation with copper ions and left to dry. Then, a Candida albicans culture was placed on the border of the film formed by the hydrogel. After 10 minutes of incubation and washing three times with saline solution, the yeast is visible mainly on the surface covered with hydrogel and is mostly washed off on the glass surface. (FIG. 8). This means that the saline solution largely washed the yeast from the glass surface, but could not do so with the yeast immobilized by the surface of the chitosan-copper hydrogel according to the invention.

C). In order to demonstrate the ability of the chitosan-copper hydrogel deposited on the surface to bind the protist Amoeba proteus, a glass microscopic coverslip was covered with a drop of hydrogel with a concentration of 10 mM and 100% saturation with copper ions and left to dry. Then, a culture was applied to the boundary of the film formed by the hydrogel and microscopic observation was performed.

Contact of the amoeba with the hydrogel film causes a rapid arrest of the amoeba, retraction of all pseudopods and cell death (FIG. 9).

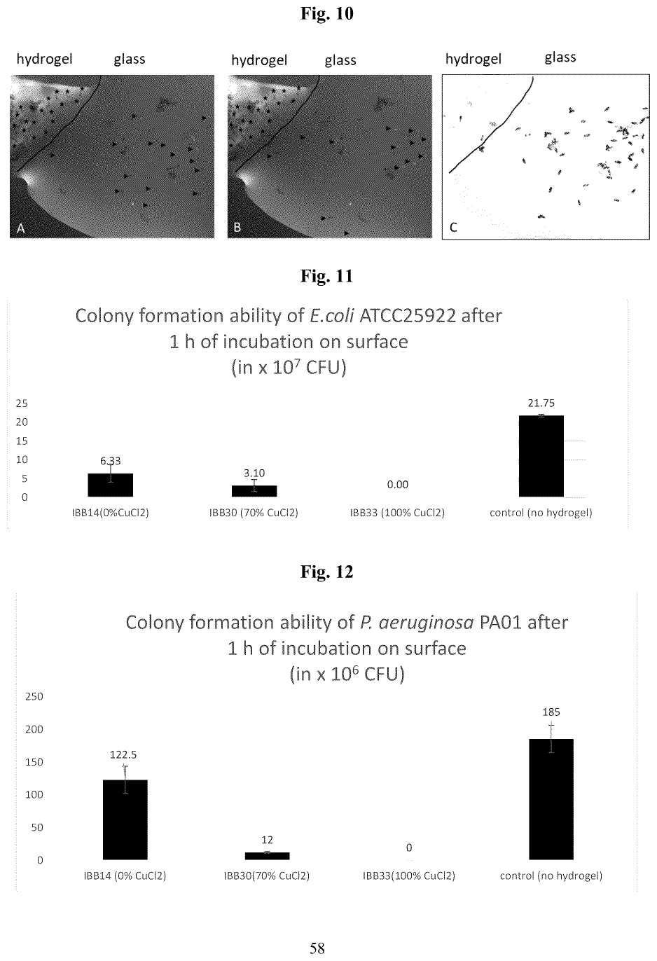

D). In order to demonstrate the ability of the chitosan-copper hydrogel deposited on the surface to bind the protists Paramecium tatraurelia, a glass microscopic coverslip was covered with a drop of hydrogel with a concentration of 10 mM and 100% saturation with copper ions and left to dry. Then, a culture of Paramecium tatraurelia was applied to the border of the film formed by the hydrogel. After 1 minute of incubation, microscopic observation was performed under visible light with Nomarski contrast (DIC). The photos were taken 1 second apart. Paramecium tatraurelia floats freely in the medium until it touches the hydrogel surface, after which it becomes immediately immobilized (FIG. 10). This means that the surface of the chitosan-copper hydrogel according to the invention prevents the migration of the protist organism, which occurs freely in the presence of a glass surface.

It is allowed to create a hydrogel film containing chitosan at the concentration of 0.01-3% and a saturation of amino groups of 20-100%. The chitosan-copper hydrogel according to the invention binds bacteria on the surface and kills them through a locally high concentration of synergistically acting biocidal chitosan and biocidal copper ions.

### Example 6. Surface Biocidal Activity of a Chitosan-Copper Hydrogel Film

A). In order to demonstrate the biocidal activity of the film covering the surface, produced by drying the hydrogel, the colony forming unit (CFU) was tested after exposing bacterial cultures to the surface covered with the hydrogel film. E. coli strain EC25922 and P. aeruginosa strain PA01 were cultured in minimal medium. The log phase culture was diluted to an amount equivalent to CFU 10³, 10⁴, 10⁵, 10⁶, 10⁷. 100 μL of culture dilutions were applied to a film of dried chitosan-copper hydrogel, prepared as in Example 1, with different saturation of amino groups. Bacterial dilutions were incubated on the hydrogel surface for 60 min. at room temperature, then harvested and sown onto solid LB medium and cultured overnight at 37° C.

The same cultures, placed on a Petri dish without hydrogel and incubated in the same conditions, were used as a control. Then, the obtained colonies on plates from individual dilutions were counted. As shown in FIG. 11, the film made of 30 mM (0.5%) of chitosan without the addition of copper(II) ions shows poor biocidal properties against E. coli bacteria. A similar effect is known from the literature. However, the film made of 30 mM (0.5%) chitosan-copper hydrogel shows a dramatic increase in biocidal activity compared to the results obtained for chitosan alone. Saturation of the hydrogel with copper(II) ions above 50% results in the complete elimination of bacterial colonies. As shown in FIG. 12, the film made of 15 mM (0.25%) of chitosan without the addition of copper(II) ions shows poor biocidal properties against P. aeruginosa bacteria. A similar effect is known from the literature. 70% saturation of the hydrogel with copper(II) ions causes a very significant reduction in the CFU value, and 100% saturation causes the complete elimination of bacterial colonies. Unlike the action in solution, the film made of the chitosan-copper hydrogel does not show any reduction in the effectiveness of the action at full, 100% saturation with copper(II) ions of the chitosan-copper hydrogel.

### Example 7. Assessment of the Formation of Chitosan-Copper Hydrogel by Examining the Viscosity of the Solution

A) Using the method of preparing the chitosan-copper hydrogel described in Example 1, solutions were obtained with a final chitosan concentration of 15 mM (0.25%) and a molar ratio of copper(II) ions to amino groups of chitosan (hereinafter referred to as saturation) equal to 5, 10, 20, 30, 40, 50, 60, 70, 80, 90, 100, 200 and 500% saturation.

The relative viscosity of the samples of the chitosan-copper hydrogels obtained in this way was measured using the flow cup method (A. M. Anderson, B. A. Bruno, L. Safford Smith, Viscosity Measurement, Chapter 23, Mechanical Engineers Handbook, ed. M. Kutz, Wiley 2015). Tab. 2 shows the results of these measurements. The relative viscosity of hydrogels according to the invention with saturation with copper(II) ions up to 50% remains constant and close to the value for chitosan itself, in the range from 60 to 100% it is significantly higher, reaching a maximum for 90 and 100%, and then decreases. When the hydrogel is saturated with copper(II) ions twice, it reaches a value close to that of the hydrogel saturated by 60%, and the viscosity of the hydrogel saturated five times is lower than that of pure chitosan.

TABLE 2
Results of determinations of the relative viscosity of chitosan-copper hydrogels with
a chitosan concentration of 15 mM (0.25%) and various saturation with copper(II) ions.

| Saturation of amino groups of chitosan | Flow time. | |
|---|---|---|
| with copper (%) | Average value of three measurements (s) | Standard deviation |
| 0 | 6.8 | 0.2 |
| 5 | 7.3 | 0.4 |
| 10 | 6.8 | 0.1 |
| 20 | 7.1 | 0.1 |
| 30 | 8 | 0.2 |
| 40 | 6.8 | 0.1 |
| 50 | 6.6 | 0.1 |
| 60 | 10.5 | 0.9 |
| 70 | 12.9 | 0.7 |
| 80 | 9.2 | 0.5 |
| 90 | 39.5 | 8.4 |
| 100 | 36.7 | 10.3 |
| 200 | 10.2 | 0.5 |
| 500 | 6.3 | 0.4 |

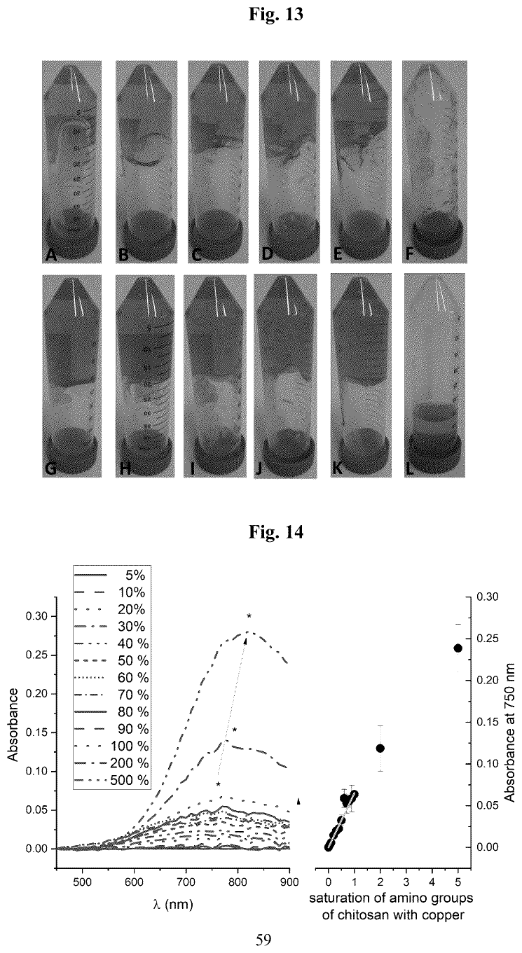

The viscosity of chitosan-copper hydrogels with higher chitosan concentration, e.g. 30 mM or 60 mM (0.5% or 1%, respectively), was too high to be determined using the above method. Instead, it was demonstrated using photographic documentation (FIG. 13), showing that as the saturation of the amino groups with copper(II) ions increases to 100%, the consistency of the hydrogel approaches that of a solid. A viscosity qualitatively higher than the viscosity of a chitosan solution of a given concentration was observed for the following ranges of saturation of amino groups of chitosan with copper(II) ions: chitosan 15 mM (0.25%): 90-100% saturation; chitosan 30 mM (0.5%): 20-100% saturation; chitosan 60 mM (1%): 10-100% saturation.

B) Using the method of preparing the chitosan-copper hydrogel described in Example 1, solutions were obtained with a final chitosan concentration of 10 mM (0.16%) and a molar ratio of copper(II) ions to amino groups of chitosan (hereinafter referred to as saturation) equal to 10, 25, 50, 75, 100% saturation. The viscosity of chitosan-copper hydrogels was tested using a digital rotational viscometer at 20° C. (Tab. 3).

TABLE 3
The viscosity level of chitosan-copper hydrogel
tested using a digital rotational viscometer.

| composition | % saturation of amino groups | Viscosity (mPa/s) |
|---|---|---|
| 10 mM chitosan | 0 | 553 |
| 10 mM chitosan 1 mM CuCl₂ | 10 | 726 |
| 10 mM chitosan 2.5 mM CuCl₂ | 25 | 860 |
| 10 mM chitosan 5 mM CuCl₂ | 50 | 1760 |
| 10 mM chitosan 7.5 mM CuCl₂ | 75 | 2330 |
| 10 mM chitosan 10 mM CuCl₂ | 100 | 2956 |

C) Using the method of preparing the chitosan-copper hydrogel described in Example 1, a solution was obtained with a final chitosan concentration of 30 mM (0.5%) and a molar ratio of copper(II) ions to amino groups of chitosan (hereinafter referred to as saturation) equal to 100% saturation. The temperature dependence of the hydrogel viscosity was examined using a digital rotational viscometer at temperatures from 10 to 90° C. (Tab. 4).

TABLE 4
The viscosity level of chitosan-copper hydrogel depending on
temperature examined using a digital rotational viscometer

| Temperature | 10 °C | 20 °C | 30 °C | 40 °C | 50 °C | 60 °C | 70 °C | 80 °C | 90 °C |
|---|---|---|---|---|---|---|---|---|---|
| Viscosity | 17500 | 16900 | 4300 | 2720 | 620 | 256 | 252 | 250 | 247 |
| (mPa/s) | | | | | | | | | |

### Example 8. Determination of the Level of Saturation of Amino Groups in the Chitosan-Copper Hydrogel by Copper(II) Ions and the Method of Binding these Ions by Examining the Absorption Spectrum in the Visible Range

Using the method of preparing chitosan-copper hydrogel described in Example 1, pt A), using chitosan at a concentration of 15 mM (0.25%) and copper (II) chloride solutions at a concentration of 1 M appropriate measured amounts of solution were added to obtain solutions with a molar ratio of copper(II) ions to amino groups of chitosan (called saturation) equal to 5, 10, 20, 30, 40, 50, 60, 70, 80, 90, 100, 200 and 500% saturation.

Samples of hydrogels obtained as described above, with a volume of 50 l were introduced into individual wells of a 96-well plate, each repeated four times. Absorption spectra of these hydrogels in the range of 400-900 nm were collected using a multifunction plate reader. The left side of FIG. 14 shows an example measurement series, and the right side shows the dependence of the absorbance intensity of these samples, averaged in the range of 700-800 nm, and then averaged over all four-measurement series. In the range from 5 to 100% saturation with copper(II) ions, the intensity of the absorption band, with a maximum at 750 nm, increases in proportion to the content of copper(II) ions in the hydrogel. Above, for 200% and 500% saturation, the absorbance of the solution increases further, but at the same time its maximum shifts towards longer wavelengths.

The linearity range of the band intensity change at 750 nm is used to assess the degree of deacetylation of amino groups in chitosan. This is important because, as shown in Example 1 pt B) and preliminary tests, batches of commercial chitosan from different suppliers have different levels of deacetylation and, therefore, different contents of amino groups. This amount should be known to properly prepare the hydrogel for specific applications. Chitosan of high purity, close to 100%, was used for the described research, which was experimentally confirmed by determining the linear range of absorbance change from 0 to 100% saturation.

The location of the band at 750 nm indicates the simultaneous binding of individual copper(II) ions in the hydrogel according to the invention to only one amino group. It follows that the principles of formation of glucosamine copper complexes, described in Micera et al. Inorg. Chim. Acta 107, 45-48, 1985, are not applicable to the binding of copper(II) ions because in the presence of glucosamine monomer, the Cu²⁺ ion at pH 7 had the ability to connect two amino sugar units through Cu—N bonds. The shift of the absorption band towards longer wavelengths when the content of copper(II) ions in the hydrogel is above 100% in relation to the amino groups indicates that the excess copper(II) ions are in the oxygen environment, in the form of aqua-ions. These excess ions break the structure of the hydrogel, which results in a decrease in its viscosity and visible delamination of its structure. Local copper(II) ions continue to interact with the chitosan chains, as evidenced by the lack of formation of copper hydroxide. Taken together, the test results presented in Examples 7 and 8 allow us to propose the structure of the hydrogel according to the invention, shown in FIG. 2B2, and its subunits in FIG. 2B1.

Due to the results obtained, for the tests in Examples 8-12, a 30 mM (0.5%) chitosan-copper hydrogel obtained using the method according to Example 1, pt A) with 50% saturation of amino groups with copper(II) ions was selected as the preferred one.

### Example 9. Testing the Ability to Create a Hydrogel from Chitosan with Various Metal Ions

The ability of salts of the following divalent metal ions to form a hydrogel with chitosan at neutral pH: Cu²⁺, Ni²⁺, Co²⁺, Zn²⁺ was tested. To 10 ml of 30 mM (0.5%) of chitosan solution obtained by the method of Example 1, pt A), 10 ml of 30 mM solutions of CuCl₂, NiCl₂, CoCl₂ hexahydrate and ZnCl₂ salts were added, respectively, obtaining a final concentration of amino groups of chitosan and metal ions of 15 mM. Only Cu²⁺ ions effectively formed a hydrogel, Zn²⁺ ions showed poor gelling properties, and Co²⁺ and Ni²⁺ ions did not show any ability to form a hydrogel.

TABLE 5
Results of relative viscosity determinations of samples containing chitosan
at a concentration of 15 mM (0.25%) and 100% saturation with metal salts.

| | Flow time. | |
|---|---|---|
| Metal salt | Average value of three measurements (s) | Standard deviation |
| none | 6.7 | 0.2 |
| CuCl₂ | 38.5 | 9 |
| NiCl₂ | 6.8 | 0.3 |
| CoCl₂ | 6.5 | 0.2 |
| ZnCl₂ | 11.4 | 0.5 |

### Example 10. Covering of Solid Surfaces with Chitosan-Copper Hydrogel

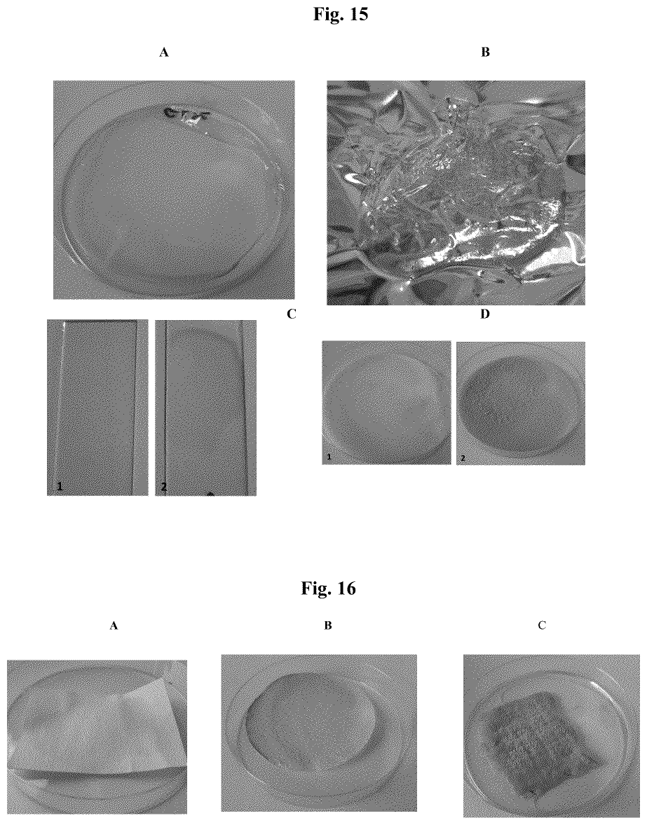

In order to demonstrate the hydrogel's ability to cover solid surfaces at room temperature, the hydrogel was applied to the surfaces of the following materials: polystyrene, glass, silica and metal (aluminum foil). Before applying the hydrogel, the polystyrene and glass surfaces were cleaned of contamination by washing with dilute nitric acid. 5 ml of 30 mM (0.5%) hydrogel, prepared according to the method described in Example 1, pt A), containing 50% saturation of amino groups with copper(II) ions, was applied to the above surfaces at room temperature, and then left to dry in a fume hood in sterile conditions. On all tested surfaces, the hydrogel formed compact thin layers, tightly adhering to the base of the dish, as shown in FIG. 15. Hydrogel concentration is allowed from 0.05 mM to 300 mM (0.0008% to 5%) depending on the required layer thickness and saturation of amino groups with copper(II) ions in the range of 5-100%.

### Example 11. Covering and Soaking of Porous Materials with Chitosan-Copper Hydrogel

5 ml of 30 mM (0.5%) chitosan-copper hydrogel prepared by the method described in Example 1, pt A) containing 50% saturation of amino groups with copper(II) was applied to a sheet of Whatman 3MM cellulose filter paper, a Whatman GF/A glass filter and a cotton fabric and then left to dry in a fume hood under sterile conditions. The hydrogel soaks into the pores of porous materials composed of glass or cellulose fibers, i.e. hydrophilic substances, creating a compact, uniform layer within them (FIG. 16). It has similar properties for linen, wool and silk fabrics and non-woven fabrics.

The durability of the covering and saturating of porous surfaces with the chitosan-copper hydrogel according to the invention has been demonstrated, which was not removed despite long-term and intensive rinsing with running water.

It is allowed to soak fabrics, non-woven fabrics and filter materials with hydrogel containing chitosan at a concentration of 0.05 mM to 300 mM (0.0008% to 5%), depending on the required layer thickness and saturation of amino groups with copper(II) ions of 5-100% in depending on the required hydrogel content in the fabric.

### Example 12. An Alternative Method of Soaking the Fabric with Chitosan-Copper Hydrogel by Using the In Situ Hydrogel Formation Method

In order to achieve more efficient coverage by penetrating the hydrogel into the fabric, e.g. cotton or glass fabric, the said fabric was sequentially soaked with chitosan and then the hydrogel was produced. For this purpose, the fabric was soaked with a 30 mM (0.5%) of chitosan solution at pH 7 (concentration from 0.05 mM to 300 mM is allowed depending on the required layer thickness). Then, a 1 M CuCl₂ solution was added to achieve 50% saturation of the amino groups with copper(II) ions (the saturation of the amino groups with copper(II) ions is allowed in the range of 5-100%), and then left to dry in a fume hood under sterile conditions. The hydrogel formed in situ in the cotton structure creates a compact, uniform layer within it. Due to the significantly higher viscosity of the chitosan-copper hydrogel than the chitosan solution of the same concentration, the penetration of the hydrogel into the fabric is difficult. Therefore, prior soaking with a chitosan solution and creating a chitosan hydrogel between the fabric fibers solves the problem of soaking porous materials with a high-viscosity hydrogel.

### Example 13. Assessment of the Durability of Surface and Fabric Coverage by Chitosan-Copper Hydrogel in the Face of Washing and Washing Processes

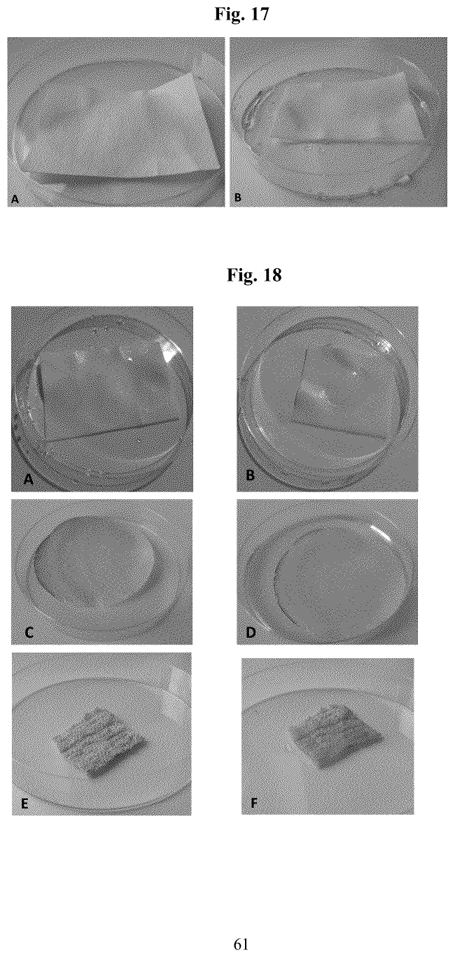

Cellulose paper (Whatman 3MM paper), glass filter (Whatman G F/A) and cotton fabric (cotton gauze) soaked with chitosan-copper hydrogel prepared according to Example 10 were exposed to a stream of tap water for a period of 5 min to recreate the process of intensive surface washing. soaked, dried and visually examined. In all three cases, the hydrogel-saturated layer remained intact (FIG. 17).

Cellulose paper, glass filter and cotton fabric soaked in the chitosan-copper hydrogel prepared according to Example 10 were incubated in a 1% sodium dodecyl sulfate (SDS) solution at room temperature for 24 h to replicate the detergent washing process, then dried and visually examined. In all three cases, the hydrogel-saturated layer remained intact despite treatment with strong detergent (FIG. 18). The durability of covering and saturating the surface with the chitosan-copper hydrogel according to the invention has been demonstrated, and it was not removed despite long-term and intensive treatment with a strong detergent.

### Example 14. Testing the Resistance of Chitosan-Copper Hydrogel to Long-Term Storage and Temperature Changes

In order to check the durability during long-term storage, a 1% hydrogel with an amino group saturation of 50%, prepared according to Example 1, pt A), was left at a temperature of 4° C. for 24 months. Unexpectedly, the hydrogel retained its biocidal activity according to the results for Example 3. It showed the ability to cover surfaces according to the results for Examples 9 and 10.

In order to check the durability of the chitosan-copper hydrogel prepared according to Example 1, pt A), it was allowed to dry (lyophilization is preferred) and stored on a shelf at room temperature for 24 months. It was then reconstituted by adding the appropriate amount of distilled water, boiling briefly and cooling. The reconstituted hydrogel did not differ from the freshly obtained material. Unexpectedly, long-term storage of the chitosan-copper hydrogel does not affect its physical and chemical properties. It is therefore possible to produce a hydrogel and obtain its dried form, e.g. in the form of granules or powder, to be reconstituted before use.

In order to check the durability of the chitosan-copper hydrogel prepared according to Example 1, pt A), it was left (without drying) at a temperature of 4° C. for 24 months in a tight container. After this time, it retained its physical and chemical properties and biocidal activity in accordance with the results from Example 3. It also showed the ability to cover surfaces in accordance with the results from Examples 9 and 10.

In order to check the durability of covering fabrics and filter materials with chitosan-copper hydrogel, prepared as in Example 1, pt A), Whatman 3MM cellulose filter paper and Whatman GF/A glass filter were covered with hydrogel as in Example 10 and left on a shelf at room temperature for 24 months. Then, the durability of the surface covering was tested in the presence of an ionic detergent bath as in Example 12. Unexpectedly, the surface saturated with hydrogel remained intact, despite long storage of the material.

In order to check the temperature dependence of the properties of the chitosan-copper hydrogel with a chitosan concentration of 0.5% (30 mM), prepared by the method from Example 1, pt A) and containing 50% saturation of amino groups with copper(II) ions, it was heated to 50° C. Unexpectedly, the viscosity of the hydrogel at this temperature dropped to the level of a chitosan solution of the same concentration, and then fully recovered after cooling the hydrogel to room temperature. Preliminary studies indicate that a decrease in the viscosity of the hydrogel can be achieved in the temperature range from 40° C. to 100° C., depending on the chitosan concentration and the saturation of the amino groups with copper ions. This means that, unexpectedly, it is possible to alternatively use the hydrogel at an elevated temperature for processes requiring its high fluidity, such as soaking porous materials or pouring it into vessels for storage or transport, and then use it at room temperature.

### Example 15. The Effect of Chitosan-Copper Hydrogel on the Oxidation of a H₂O₂-Sensitive Protein

In order to demonstrate the mechanism of the biocidal action of the chitosan-copper hydrogel, its influence on the oxidation state of the HyPer7 fluorescent protein was examined. This protein shows different fluorescence profiles in the reduced and oxidized states, which can be determined by recording the ratio of the fluorescence emission intensity at 520 nm when excited at 485 nm (oxidized protein) to the fluorescence at 520 nm when excited at 400 nm (reduced protein) (Pak et al. Cell Metabolism 31, 642-653, 2020). Values of the R_{485/400} ratio below 0.6 mean completely reduced protein, and above 1.8, completely oxidized protein. As shown in FIG. 19, under the experimental conditions, the HyPer7 protein in the buffer without and with the addition of 1 mM of chitosan undergoes slow oxidation, which is prevented, as expected, by the presence of DTT reducing agent. In the presence of 10 mM H₂O₂, the HyPer7 protein is rapidly oxidized. Chitosan-copper hydrogel with a concentration of 1 mM and a degree of copper(II) saturation of 25% or higher caused protein oxidation to an extent comparable to 10 mM H₂O₂, even though the only oxidant in these reactions was atmospheric oxygen. Only in the presence of chitosan-copper hydrogel with a saturation level of 10% was the reaction significantly slower, but asymptotically it tended to complete protein oxidation. This means that the chitosan-copper hydrogel has strong oxidizing abilities, without the need of adding chemical oxidants to it.

### Example 16. The Effect of Chitosan-Copper Hydrogel on the Oxidation of an H₂O₂-Sensitive Protein Inside a Cell of Escherichia coli Bacterium

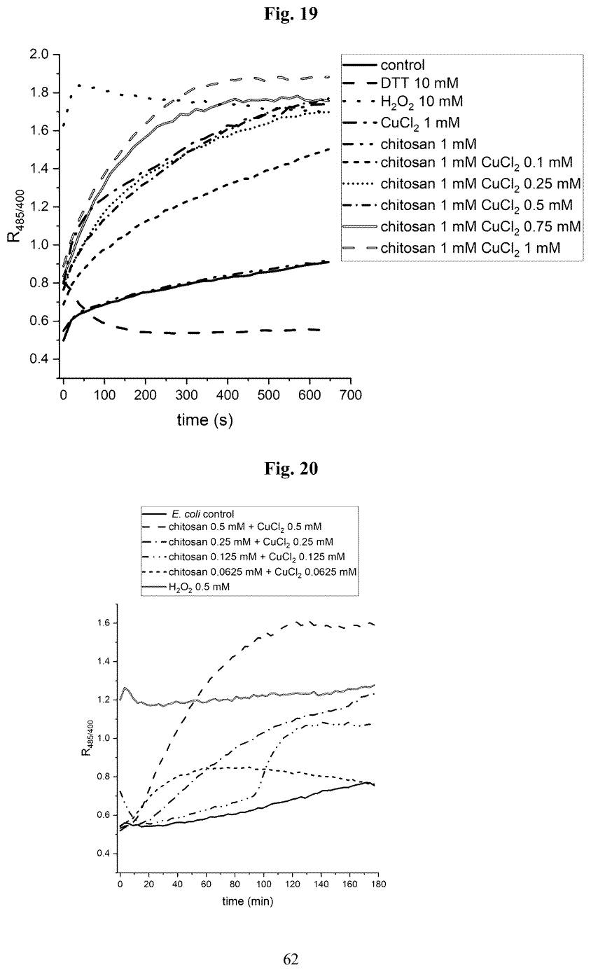

In order to demonstrate the mechanism of action of the biocidal activity of the chitosan-copper hydrogel according to the invention, an analogous series of experiments was performed to determine the level of oxidative stress in E. coli bacteria overexpressing the HyPer7 protein, which is a marker of the H₂O₂ level. E. coli bacteria overexpressing the fluorescent HyPer7 protein were cultured in minimal medium (doi:10.1101/pdb.rec12295 Cold Spring Harb Protoc 2010). The starter culture was diluted to OD=0.1 and grown with shaking in a 96-well plate, fluorescence was measured every 3 min for 3 h. The volume of a single well was 100 μl. Culture was performed and fluorescence was measured in a universal plate reader (FLUOstar Omega, BMG Labtech). The culture was carried out in the presence of chitosan-copper hydrogel with various degrees of hydrogel saturation with copper and in various dilutions. This protein shows different fluorescence profiles in the reduced and oxidized states, which can be determined by recording the ratio of the fluorescence emission intensity at 520 nm when excited at 485 nm (oxidized protein) to the fluorescence at 520 nm when excited at 400 nm (reduced protein). (Pak et al. Cell Metabolism 31, 642-653, 2020). Values of the R_{485/400} ratio below 0.6 mean a completely reduced protein, and above 1.8, a completely oxidized protein. The HyPer7 protein is located inside the bacterial cell and the oxidation of this protein indicates an increased level of H₂O₂ inside the bacterial cell. FIG. 20 shows the change in the oxidation level of the HyPer7 protein over time. In the control culture, the HyPer7 protein is in a reduced state and in the presence of 0.5 mM H₂O₂ it is significantly oxidized. Cultivation in the presence of 0.5 mM of chitosan does not change the oxidation level of the HyPer7 protein. Cultivation in the presence of 0.5 mM CuCl₂ causes an increase in the level of oxidized HyPer7 protein comparable to culture in the presence of chitosan-copper hydrogel at a concentration of 0.5 mM of chitosan, 0.125 mM CuCl₂ (25% saturation). Culture in the presence of chitosan-copper hydrogel at a concentration of 0.5 mM of chitosan, 0.25 mM CuCl₂ (50% saturation) and higher causes complete oxidation of the HyPer7 protein in bacterial cells.

The effect of chitosan-copper hydrogel on the oxidation of the HyPer7 protein depends on the hydrogel concentration, with the same degree of saturation with cupric ions (FIG. 20). For the 100% saturation used, the hydrogel with a concentration in the culture of 0.5 mM causes oxidative stress in bacteria that is stronger than the same concentration of H₂O₂, the activity of the hydrogel with a concentration twice as low is proportionally lower, and further dilutions of the hydrogel cause a gradual delay and disappearance of the effect in the tested bacteria.

### Example 17. Effect of Chitosan-Copper Hydrogel on Lipid Peroxidation in Escherichia coli

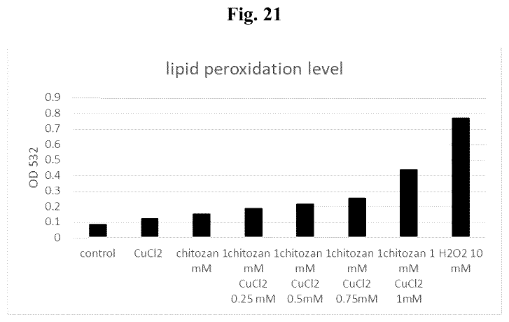

In order to demonstrate the mechanism of the biocidal action of the chitosan-copper hydrogel, its effect on the degree of lipid peroxidation in Escherichia coli was examined. Bacteria were grown in minimal medium (doi:10.1101/pdb.rec12295 Cold Spring Harb Protoc 2010). The starting culture was diluted to OD=0.2 and cultured with shaking in a volume of 2 ml for 1 hour in the presence of a hydrogel with different saturation with Cu(II) ions. Lipid peroxidation was determined (using Merck kit code MAK085-1kit) by reacting MDA with thiobarbituric acid (TBA) and generating a colorimetric reaction with an absorbance maximum at 532 nm. FIG. 21 shows the level of lipid peroxidation.

1. Chitosan-copper hydrogel characterized in that it contains
from 0.0008 mM to 300 mM of chitosan with a degree of chitosan deacetylation of at least 20%, wherein the degree of deacetylation quantitatively corresponds to the content of the reactive amino groups of chitosan;
wherein the reactive amino groups of chitosan are saturated with copper(II) ions in an amount corresponding to from 5 to 100% of the total content of the reactive amino groups of chitosan;
wherein the chitosan-copper hydrogel has pH 7±1, preferably pH 7,
wherein the chitosan-copper hydrogel has a liquid form at the temperature above 80° C.

2. Chitosan-copper hydrogel according to claim 1, characterized in that it is produced by basic subunits I, basic subunits II, and basic subunits III, wherein
the basic subunits I are non-deacetylated glucosamine subunits of chitosan, with the formula (I) 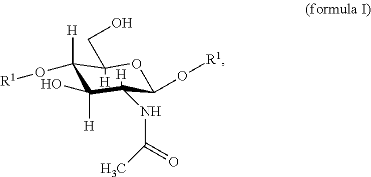
wherein the basic subunits II are deacetylated chitosan glucosamine subunits with the reactive amino groups of chitosan unsaturated with copper(II) ions, with the formula (II) 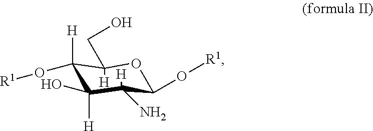
wherein the basic subunits III are deacetylated chitosan glucosamine subunits with the reactive amino groups of chitosan saturated with copper(II) ions, with the formula (III) 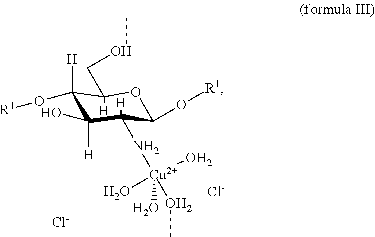
wherein the basic subunits I, II, III are linked together in any order, and wherein R¹ in the formula I, II, III is the chain composed of these basic subunits I, II, III.

3. Chitosan-copper hydrogel according to claim 2, characterized in that it contains from 0.6 mM to 180 mM of chitosan.

4. Chitosan-copper hydrogel according to claims 1-3, characterized in that the degree of chitosan deacetylation is at least 30%, 40%, 50%, 60%, 70%, 80%, 90%, 95%, 98%, 99%, most preferably 100%.

5. Chitosan-copper hydrogel according to claims 1-4, characterized in that the amino groups of chitosan are saturated with copper(II) ions in an amount corresponding to from 10 to 100%, preferably from 20% to 95%, preferably from 30 to 90%, preferably from 40 to 80%, preferably from 50 to 100%, most preferably from 50 to 70% of saturation of the initially reactive amino groups of chitosan.

6. Chitosan-copper hydrogel according to claims 1-5, characterized in that it is in a dried form, preferably in a freeze-dried form, preferably in the form of a powder, granulate, preferably in a dried form ready to be reconstituted by adding water.

7. A method for producing a chitosan-copper hydrogel, characterized in that it includes the steps in which:
a) chitosan with a degree of deacetylation of at least 20%, wherein the degree of deacetylation quantitatively corresponds to the content of the reactive amino groups of chitosan, dissolved in a water-soluble inorganic and/or organic acid at a concentration of 0.1% to 10% w/v, preferably 1% w/v of the acid, to obtain a chitosan solution with a concentration of 0.0008 mM to 300 mM;
preferably, dissolution of chitosan is carried out at 10-80° C., preferably at 20-30° C.;
b) the solution of dissolved chitosan from the step a) is separated from the acid and adjusted to pH 7±1 by dialysis in water, preferably distilled water, preferably at the temperature of 20-30° C.;
c) an aqueous solution of an inorganic or organic copper(II) salt is added to the solution from the step b) while stirring vigorously, and the copper(II) salt solution is added in an amount corresponding to a molar ratio in terms of copper(II) ions corresponding to from 5 to 100% saturation of the reactive amino groups of chitosan, preferably at the temperature of 20-30° C.;
wherein the produced chitosan-copper hydrogel has pH 7±1, preferably has pH 7, and wherein the chitosan-copper hydrogel has a liquid form at the temperature above 80° C.

8. The method for producing chitosan-copper hydrogel according to claim 7, characterized in that in the step a) from 0.6 mM to 180 mM of chitosan is used.

9. The method for producing chitosan-copper hydrogel according to claims 7-8, characterized in that in the step a) chitosan with a degree of deacetylation of at least 30%, 40%, 50%, 60%, 70%, 80%, 90%, 95%, 98%, 99%, most preferably 100% is used.

10. The method for producing chitosan-copper hydrogel according to claims 7-9, characterized in that in the step d) an aqueous solution of an inorganic or organic copper(II) salt is added in an amount corresponding to a molar ratio in terms of copper(II) ions, corresponding to from 10 to 100%, preferably from 20% to 95%, preferably from 30 to 90%, preferably from 40 to 80%, preferably from 50 to 100%, most preferably from 50 to 70% saturation of the reactive amino groups of chitosan.

11. The method for producing chitosan-copper hydrogel according to claims 7-10, characterized in that in step a) the acid is an inorganic acid selected from hydrochloric acid, sulfuric acid, nitric acid, perchloric acid and/or an organic acid selected from carboxylic acid, hydroxycarboxylic acid, sulfonic acid, acetic acid, trifluoroacetic acid, glycolic acid, lactic acid, mandelic acid, formic acid, salicylic acid, or a mixture thereof, more preferably the acid is acetic acid.

12. The method for producing chitosan-copper hydrogel according to claims 7-11, characterized in that in the step c) the aqueous solution of the inorganic or organic copper(II) salt is an aqueous solution of copper(II) chloride, sulfate, nitrate, perchlorate, acetate, trifluoroacetate, lactate, glycolate, formate, salicylate or mandelate.

13. A method for producing chitosan-copper hydrogel, characterized in that it includes the steps
a) chitosan with a degree of chitosan deacetylation of at least 20%, wherein the degree of deacetylation corresponds quantitatively to the content of the reactive amino groups of chitosan, is dissolved in a water-soluble inorganic and/or organic acid at a concentration of 0.1% to 10%, w/v, preferably 1% w/v of acid, to obtain a chitosan solution of 0.05 mM to 300 mM;
wherein, preferably, from 0.6 mM to 180 mM of chitosan is used;
wherein, preferably, chitosan with a degree of deacetylation of at least 30%, 40%, 50%, 60%, 70%, 80%, 90%, 95%, 98%, 99%, the most preferably 100% is used;
wherein, preferably, the acid is an inorganic acid selected from hydrochloric acid, sulfuric acid, nitric acid, perchloric acid and/or an organic acid selected from carboxylic acid, hydroxycarboxylic acid, sulfonic acid, acetic acid, trifluoroacetic acid, glycolic acid, lactic acid, mandelic acid, formic acid, salicylic acid, or a mixture thereof, more preferably the acid is acetic acid,
wherein the dissolution of chitosan is preferably carried out at the temperature of 10-80° C., preferably at the temperature of 20-30° C.;
b) an aqueous solution of an inorganic or organic copper(II) salt is added to the solution from the step a) while stirring, and the copper(II) salt solution is added in an amount corresponding to from 5 to 100% of saturation of the reactive amino groups of chitosan, preferably at the temperature of 20-30° C.;
more preferably, an aqueous solution of an inorganic or organic copper(II) salt is added in an amount corresponding to from 10 to 100%, preferably from 20% to 95%, preferably from 30 to 90%, preferably from 40 to 80%, preferably from 50 to 100%, most preferably from 50 to 70% of saturation of the reactive amino groups of chitosan;
more preferably, the aqueous solution of an inorganic or organic copper(II) salt is an aqueous solution of copper(II) chloride, sulfate, nitrate, perchlorate, acetate, trifluoroacetate, lactate, glycolate, formate, salicylate, or mandelate;
c) the mixture as obtained in the step b) is contacted with a solution of a base or a buffer not containing agents chelating cupric ions, preferably the base is added to the solution to obtain pH 7±1, more preferably pH 7, wherein preferably the base is selected from NaOH, KOH, preferably at the temperature of 20-30° C.,
wherein the produced chitosan-copper hydrogel has pH 7±1, preferably has pH 7, and wherein the chitosan-copper hydrogel has a liquid form at the temperature above 80° C.

14. The method for producing chitosan-copper hydrogel according to claim 13, characterized in that the chitosan-copper hydrogel is formed in situ on the surface to be covered with the chitosan-copper hydrogel, wherein
between the step b) and c) the mixture produced in the step b) is contacted with the surface on which the hydrogel is to be produced, preferably the surface is selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface is hydrophilic and/or negatively charged, and the contacting leads to by applying, spreading, spraying and/or immersing the surface in the mixture produced in the step b).

15. A method for producing chitosan-copper hydrogel, characterized in that it includes the steps
a) chitosan with a degree of chitosan deacetylation of at least 20%, wherein the degree of deacetylation corresponding quantitatively to the content of the reactive amino groups of chitosan;
is dissolved in a water-soluble inorganic and/or organic acid at a concentration of 0.1% to 10% w/v, to obtain a solution of 0.0008 mM to 300 mM;
wherein from 0.6 mM to 180 mM of chitosan is preferably used;
wherein chitosan with a degree of deacetylation of at least 30%, 40%, 50%, 60%, 70%, 80%, 90%, 95%, 98%, 99%, most preferably 100% is used;
wherein, preferably, the acid is an inorganic acid selected from hydrochloric acid, sulfuric acid, nitric acid, perchloric acid and/or an organic acid selected from carboxylic acid, hydroxycarboxylic acid, sulfonic acid, acetic acid, trifluoroacetic acid, glycolic acid, lactic acid, mandelic acid, formic acid, salicylic acid, or a mixture thereof, more preferably the acid is acetic acid;
wherein the dissolution of chitosan is preferably carried out at the temperature of 10-80° C., preferably at the temperature of 20-30° C.;
b) to the solution obtained in a) a solution of base or buffer not containing agents chelating cupric ions is added to obtain pH 7±1, preferably the base is selected from NaOH, KOH, preferably at the temperature of 20-30° C.;
c) the mixture obtained in step b) is contacted with, preferably added to, an aqueous solution of an inorganic or organic copper(II) salt while stirring, and the copper(II) salt solution is added in an amount corresponding to from 5 to 100% of saturation of the reactive amine groups of chitosan; preferably at the temperature of 20-30° C.;
more preferably, an aqueous solution of an inorganic or organic copper(II) salt is added in an amount corresponding to from 10 to 100%, preferably from 20% to 95%, preferably from 30 to 90%, preferably from 40 to 80%, preferably from 50 to 100%, most preferably from 50 to 70% of saturation of the reactive amino groups of chitosan;
more preferably, the aqueous solution of an inorganic or organic copper(II) salt is an aqueous solution of copper(II) chloride, sulfate, nitrate, perchlorate, acetate, trifluoroacetate, lactate, glycolate, formate, salicylate, or mandelate;
wherein the produced chitosan-copper hydrogel has pH 7±1, preferably has pH 7, and wherein the chitosan-copper hydrogel has a liquid form at the temperature above 80° C.

16. The method for producing chitosan-copper hydrogel according to claim 15, characterized in that the chitosan-copper hydrogel is produced in situ on the surface to be covered with the chitosan-copper hydrogel, and between the step b) and c) the mixture produced in the step b) is contacted with the surface on which the hydrogel is to be produced, wherein the surface is preferably selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface is hydrophilic and/or negatively charged in nature, and contacting is carried out by applying, spreading, spraying and/or immersing the surface in the mixture prepared in the step b).

17. The method of covering a surface with the chitosan-copper hydrogel as defined in claim 1-6 and/or produced by the method as defined in claim 7-12 and/or by the method as defined in claim 13-14 and/or by the method as defined in 15-16, wherein
the surface being covered is contacted with the chitosan-copper hydrogel, wherein the contacting is carried out by overlaying, applying, spraying the chitosan-copper hydrogel onto the surface and/or immersing the surface in the chitosan-copper hydrogel, and drying the surface covered with the chitosan-copper hydrogel;
wherein the surface is preferably selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface has a hydrophilic character.

18. The method of covering the surface with chitosan-copper hydrogel according to claim 17, characterized in that the chitosan-copper hydrogel is heated to the temperature of 40-100° C., preferably 50-80° C., preferably 50° C. before contacting the surface to be covered.

19. A surface of an object characterized in that it is covered with a chitosan-copper hydrogel as defined in claims 1-6 and/or prepared by the method as defined in claims 7-12 and/or prepared by the method as defined in claims 13-14 and/or prepared by the method as defined in claim 13-14 and/or prepared by the method as defined in claim 15-17 and/or prepared by the method as defined in claims 17-18,
wherein the surface of the object is preferably selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface has a hydrophilic and/or negatively charged character.

20. A fabric or non-woven fabric characterized in that it is covered with the chitosan-copper hydrogel as defined in claims 1-5 and/or prepared by the method as defined in claims 7-12 and/or prepared by the method as defined in claims 13-14 and/or prepared by the method as defined in claims 15-16 and/or prepared with the method as defined in claims 17-18,
wherein the fabric or non-woven fabric is made of cotton, linen, wool, silk, polyester, polylactide, aromatic-aliphatic polyesters, polystyrene or a mixture thereof.

21. A composition for covering surfaces to prevent the development, to inhibit the growth and kill bacteria, fungi, viruses, protozoa, characterized in that it contains the chitosan-copper hydrogel as defined in claims 1-6 and/or prepared by the method as defined in claims 7-12 and/or prepared by the method as defined in claims 13-14 and/or prepared by the method as defined in claims 15-16,
wherein the surface is preferably selected from paper, cellulose, cellulose paper, glass, soda glass, glass non-woven fabric, cotton, linen, wool, silk, metal, polyester, polylactide, aromatic-aliphatic polyesters, polystyrene, fabric, non-woven fabric, iron, cast iron, metal, aluminum, steel, stainless steel, galvanized steel, alloy steel, copper, bronze, brass, preferably the surface has a hydrophilic character.

22. A pharmaceutical composition characterized in that it contains the chitosan-copper hydrogel as defined in claims 1-6 and/or prepared by the method as defined in claims 7-12 and/or prepared by the method as defined in claims 13-14 and/or prepared by the method as defined in claims 15-16, wherein the composition further comprises at least one pharmaceutically acceptable carrier.

23. The pharmaceutical composition of claim 22, characterized in that it is in the form of a liquid, emulsion, gel, spray, foam, nasal spray, mouthwash, hand wash gel, wet wipe or dressing, preferably the dressing is in the form of a patch with hydrogel, gauze with the composition applied, hydrocolloid dressing, hydrofibrous dressing, bandage with the composition applied.

24. A pharmaceutical composition containing the chitosan-copper hydrogel as defined in claims 1-6 and/or prepared by the method as defined in claims 7-12 and/or prepared by the method as defined in claims 13-14 and/or prepared by the method as defined in claims 15-16, for use as a medicine for the treatment of diseases and/or inflammations caused by bacteria, fungi, viruses, protozoa, preferably against bacteria of the genus Escherichia, Pseudomonas, Staphylococcus, Enterococcus, Acinetobacter, Proteus, fungi of the genus Candida, retroviruses, lentiviruses.

25. A cosmetic or care composition for cosmetic, care and hygiene applications in humans and/or animals, characterized in that the chitosan-copper hydrogel as defined in claims 1-6 and/or prepared by the method as defined in claims 7-12 and/or prepared by the method as defined in claims 13-14 and/or produced by the method as defined in claims 15-16, wherein the composition additionally contains at least one carrier approved for cosmetic, care and hygiene applications in humans and/or animals and is intended for external use.

26. The cosmetic or care composition according to claim 25, characterized in that it is in the form of a liquid, emulsion, gel, cream, spray, hand washing gel, spray, wet wipe.

27. A composition comprising the chitosan-copper hydrogel as defined in claim 16 and/or prepared by the method as defined in claims 7-12 and/or prepared by the method as defined in claim 13-14 and/or prepared by the method as defined in claims 15-16, for external use as a biocide, antiseptic, antibacterial agent, antiviral agent, fungicide, surface disinfectant, wherein, preferably, the composition is used against bacteria of the genus Escherichia, Pseudomonas, Staphylococcus, Enterococcus, Acinetobacter, Proteus, fungi of the genus Candida, retroviruses, lentiviruses, protozoa.

28. The composition according to claim 27, characterized in that it is in the form of a liquid, emulsion, gel, spray, lotion, wet wipe, paper towel, handkerchief, hygiene material.

29. A veterinary composition characterized in that it contains the chitosan-copper hydrogel as defined in claims 1-6 and/or prepared by the method as defined in claims 7-12 and/or prepared by the method as defined in claims 13-14 and/or prepared by the method as defined in claims 15-16, wherein the composition additionally comprises at least one carrier approved for veterinary use, wherein the composition is preferably in the form of a liquid, emulsion, gel, spray, foam, spray, liquid, cleansing gel, wet wipe or dressing, preferably the dressing is in the form of a patch with hydrogel, gauze with the composition applied, hydrocolloid dressing, hydrofibrous dressing, bandage with the composition applied.

30. A veterinary composition comprising the chitosan-copper hydrogel as defined in claims 1-6 and/or prepared by the method as defined in claims 7-12 and/or prepared by the method as defined in claims 13-14 and/or prepared by the method as defined in claims 15-16, for use as a medicine for the treatment of diseases and/or inflammations caused by bacteria, fungi, viruses, protozoa, preferably against bacteria of the genus Escherichia, Pseudomonas, Staphylococcus, Enterococcus, Acinetobacter, Proteus, fungi of the genus Candida, retroviruses, lentiviruses.

31. Use of the chitosan-copper hydrogel as defined in claims 1-6 and/or prepared by the method as defined in claims 7-21 and/or prepared by the method as defined in claims 13-14 and/or prepared by the method as defined in claims 15-16 as an externally applied biocidal agent, antiseptic agent, antibacterial agent, bacteriostatic agent, antiviral agent, fungicide, agent for covering, soaking and disinfecting surfaces,
preferably against bacteria of the genus Escherichia, Pseudomonas, Staphylococcus, Enterococcus, Acinetobacter, Proteus, fungi of the genus Candida, retroviruses, lentiviruses, protozoa.

32. The use according to claim 31, characterized in that the agent is used in health care to cover and soak the surfaces of fabrics, tools and devices used in medicine, veterinary medicine and diagnostics, and other surfaces, in particular hospital and laboratory surfaces, surfaces in public places, medical and veterinary clinics, handrails, door handles, toilets, taps, sanitary facilities, floors, walls.

33. The use according to claim 31, characterized in that the agent is used in the textile industry for covering fabrics and non-woven fabrics, preferably for covering medical fabrics and non-woven fabrics, preferably for covering fabrics and non-woven fabrics used to produce filters, air filters, medical textiles, gauze, dressings, bandages, masks protective clothing, medical gowns, protective suits, hospital mattresses, quilts, pillows, bedding, hospital bedding.

34. The use according to claim 31, characterized in that the agent is used in the cosmetics industry, preferably as an additive to cosmetics improving their microbiological quality, preferably as an additive to liquids, foams, gels, creams, milks, lotions.

35. The use according to claim 32, characterized in that the agent is used as a biocidal agent, antiseptic agent, antibacterial agent, bacteriostatic agent, antiviral agent, fungicide in the form of a liquid, emulsion, gel, foam, spray liquid, lotion, wet wipe or dressing, wherein it is preferably used as a bacteriostatic or bactericidal agent in the form of a plaster with a dressing, a patch with a hydrogel, gauze with the composition applied, a dressing, a bandage with the composition applied.

36. Use of the chitosan-copper hydrogel as defined in claims 1-6 and/or produced by the method as defined in claims 7-12 and/or produced by the method as defined in claims 13-14 and/or produced by the method as defined in claims 15-16, as an agent for purifying fluids from biological contaminants in the form of bacteria, fungi, viruses, protozoa by aggregation and inhibiting the growth of bacteria, fungi, viruses, protozoa on chitosan-copper hydrogel and sedimentation, preferably the purified fluids are biologically polluted waters, preferably waters in water treatment plants.

37. A method for purifying aqueous solutions from microbiological contaminants in the form of bacteria, fungi, viruses, protozoa, characterized in that the chitosan-copper hydrogel as defined in claims 1-6 and/or prepared by the method as defined in claims 7-12 and/or prepared by the method as defined in claims 13-14 and/or produced by the method as defined in claims 15-16 is added to the contaminated aqueous solution;
after a pause, preferably with stirring, for the formation of aggregates of microorganisms on the chitosan-copper hydrogel,
the formed aggregates are isolated from the purified aqueous solution, preferably they are isolated by sedimentation.
